SEQUENCE LISTING (74 sequences)

SEQ ID NO: 1 - RNA, 25 nt, Human
uuccuaaagagaaaagagauaugaa

SEQ ID NO: 2 - RNA, 20 nt, Human
ccaucgucuuccgaugguau

SEQ ID NO: 3 - RNA, 25 nt, Human
gcuguaaccguggagagcagcaccg

SEQ ID NO: 4 - RNA, 25 nt, Human
acacgccccuggaaacugggcugua

SEQ ID NO: 5 - RNA, 22 nt, Human
gggcaugcagaauuccuucuac

SEQ ID NO: 6 - RNA, 25 nt, Human
accuuuuggauaguugacaacauac

SEQ ID NO: 7 - RNA, 25 nt, Human
auccauuuuucuuuuguuaucuaaa

SEQ ID NO: 8 - RNA, 25 nt, Human
cuuucacugcugaugaagcaucugu

SEQ ID NO: 9 - RNA, 25 nt, Human
aacacuauuaaaucggauguuuucu

SEQ ID NO: 10 - RNA, 25 nt, Human
cagguccuguucgguuguuuuccau

SEQ ID NO: 11 - RNA, 25 nt, Human
uuuaccugcaccauaagaacuucuc

SEQ ID NO: 12 - RNA, 25 nt, Human
aaccguggagagcagcaccguccca

SEQ ID NO: 13 - RNA, 25 nt, Human
acugggcuguaaccguggagagcag

SEQ ID NO: 14 - RNA, 25 nt, Human
uggaaacugggcuguaaccguggag

SEQ ID NO: 15 - RNA, 25 nt, Human
acugcugaugaagcaucugucucau

SEQ ID NO: 16 - RNA, 25 nt, Human
uuucacuuucacugcugaugaagca

SEQ ID NO: 17 - RNA, 25 nt, Human
uauuaaaucggauguuuucuggacu

SEQ ID NO: 18 - RNA, 25 nt, Human
gacccaacacuauuaaaucggaugu

SEQ ID NO: 19 - RNA, 25 nt, Human
ccuguucgguuguuuuccaugggag

SEQ ID NO: 20 - RNA, 25 nt, Human
ucuuucagguccuguucgguuguuu

SEQ ID NO: 21 - RNA, 20 nt, Human
caguuuuauuuaagaauuuc

SEQ ID NO: 22 - RNA, 28 nt, Human
guuuuauuuaagaauuuccaacaaugac

SEQ ID NO: 23 - RNA, 30 nt, Human
gacacauauuauuuaaaucaguuuuauuua

SEQ ID NO: 24 - DNA, 25 bp, Human
aaccgtggagagcagcaccgtccca

SEQ ID NO: 25 - DNA, 25 bp, Human
tttcactttcactgctgatgaagca

SEQ ID NO: 26 - RNA, 20 nt, Human
caccuuccuucuuuuugauu

SEQ ID NO: 27 - RNA, 25 nt, Human
ggauguccugagucuagacccuccg

SEQ ID NO: 28 - DNA, 25 bp, Human
cctcttacctcagttacaatttata

SEQ ID NO: 29 - RNA, 25 nt, Human
cgaaggucuucugccgaguccugca

SEQ ID NO: 30 - RNA, 25 nt, Human
cauucacauucauuucuucuuaggc

SEQ ID NO: 31 - RNA, 25 nt, Human
uuuguuuuaauuucuucaguauugc

SEQ ID NO: 32 - RNA, 25 nt, Human
gucuucugccgaguccugcaauaug

SEQ ID NO: 33 - RNA, 25 nt, Human
ucucucgaaggucuucugccgaguc

SEQ ID NO: 34 - RNA, 25 nt, Human
acauucauuucuucuuaggcaggcu

SEQ ID NO: 35 - RNA, 25 nt, Human
agccgcauucacauucauuucuucu

SEQ ID NO: 36 - DNA, 25 bp, Human
tctctcgaaggtcttctgccgagtc

SEQ ID NO: 37 - DNA, 25 bp, Human
agccgcattcacattcatttcttct

SEQ ID NO: 38 - DNA, 25 bp, Human
ttcctaaagagaaaagagatatgaa

SEQ ID NO: 39 - DNA, 20 bp, Human
ccatcgtcttccgatggtat

SEQ ID NO: 40 - DNA, 25 bp, Human
gctgtaaccgtggagagcagcaccg

SEQ ID NO: 41 - DNA, 25 bp, Human
acacgcccctggaaactgggctgta

SEQ ID NO: 42 - DNA, 22 bp, Human
gggcatgcagaattccttctac

SEQ ID NO: 43 - DNA, 25 bp, Human
accttttggatagttgacaacatac

SEQ ID NO: 44 - DNA, 25 bp, Human
atccatttttcttttgttatctaaa

SEQ ID NO: 45 - DNA, 25 bp, Human
ctttcactgctgatgaagcatctgt

SEQ ID NO: 46 - DNA, 25 bp, Human
aacactattaaatcggatgttttct

SEQ ID NO: 47 - DNA, 25 bp, Human
caggtcctgttcggttgttttccat

SEQ ID NO: 48 - DNA, 25 bp, Human
tttacctgcaccataagaacttctc

SEQ ID NO: 49 - DNA, 25 bp, Human
actgggctgtaaccgtggagagcag

SEQ ID NO: 50 - DNA, 25 bp, Human
tggaaactgggctgtaaccgtggag

SEQ ID NO: 51 - DNA, 25 bp, Human
actgctgatgaagcatctgtctcat

SEQ ID NO: 52 - DNA, 25 bp, Human
tattaaatcggatgttttctggact

SEQ ID NO: 53 - DNA, 25 bp, Human
gacccaacactattaaatcggatgt

SEQ ID NO: 54 - DNA, 25 bp, Human
cctgttcggttgttttccatgggag

SEQ ID NO: 55 - DNA, 25 bp, Human
tctttcaggtcctgttcggttgttt

SEQ ID NO: 56 - DNA, 20 bp, Human
cagttttatttaagaatttc

SEQ ID NO: 57 - DNA, 28 bp, Human
gttttatttaagaatttccaacaatgac

SEQ ID NO: 58 - DNA, 30 bp, Human
gacacatattatttaaatcagttttattta

SEQ ID NO: 59 - DNA, 20 bp, Human
caccttccttctttttgatt

SEQ ID NO: 60 - DNA, 25 bp, Human
ggatgtcctgagtctagaccctccg

SEQ ID NO: 61 - DNA, 25 bp, Human
cctcttacctcagttacaatttata

SEQ ID NO: 62 - DNA, 25 bp, Human
cgaaggtcttctgccgagtcctgca

SEQ ID NO: 63 - DNA, 25 bp, Human
cattcacattcatttcttcttaggc

SEQ ID NO: 64 - DNA, 25 bp, Human
tttgttttaatttcttcagtattgc

SEQ ID NO: 65 - DNA, 24 bp, Human
gtctctgccgagtcctgcaatatg

SEQ ID NO: 66 - DNA, 25 bp, Human
acattcatttcttcttaggcaggct

SEQ ID NO: 67 - DNA, 20 bp, Human
cattttgtgggagcgaagcg

SEQ ID NO: 68 - DNA, 20 bp, Human
acgcaccaaagttcatccca

SEQ ID NO: 69 - DNA, 20 bp, Human
agcgcaagggtttctggttt

SEQ ID NO: 70 - DNA, 20 bp, Human
ggagtcatgggggagggata

SEQ ID NO: 71 - DNA, 20 bp, Human
tgtactccagcaccattggc

SEQ ID NO: 72 - DNA, 21 bp, Human
aaagttcgtggggcttctgag

SEQ ID NO: 73 - DNA, 20 bp, Human
tgcttcagccagacagttca

SEQ ID NO: 74 - DNA, 19 bp, Human
gtcaactgtgccacaagcc

(19) United States
(12) Patent Application Publication
(10) Pub. No.: US 2025/0270548 A1
(43) Pub. Date: Aug. 28, 2025

(54) COMPOSITIONS AND METHODS FOR TREATING TARDBP ASSOCIATED DISEASES

(71) Applicant: PERRON INSTITUTE FOR NEUROLOGICAL AND TRANSLATIONAL SCIENCE LIMITED, Nedlands, Western Australia (AU)

(72) Inventors: Rita Mejzini, Yangebup (AU); Patrick Anthony Akkari, Mullaloo (AU); Loren Louise Flynn, Karrinyup (AU); Stephen Donald Wilton, Applecross (AU); Sue Fletcher, Bayswater (AU)

(21) Appl. No.: 18/039,928

(22) PCT Filed: Nov. 8, 2021
(86) PCT No.: PCT/AU2021/051318

§ 371 Date: Jun. 1, 2023

(30) Foreign Application Priority Data
Dec. 7, 2020 (AU) 2020904531

(51) Int. Cl. C12N 15/113

(52) U.S. Cl. CPC C12N 15/113; C12N 2310/11; C12N 2310/315; C12N 2310/321

(57) ABSTRACT

The present invention relates to the field of antisense oligonucleotides used to reduce expression of the transactive response DNA binding protein 43 (TARDBP) gene which encodes the protein TAR DNA-binding protein 43 (TDP43). The present invention also relates to the field of antisense oligonucleotides used to prevent the subsequent downregulation of stathmin-2. The invention also provides pharmaceutical compositions and methods to treat the effects of a disease associated with TDP43 proteinopathy by administration of antisense oligonucleotides and therapeutic compositions comprising AONs targeted to TARDBP and AONs targeted to STMN2.

### TECHNICAL FIELD

The present invention relates to antisense oligonucleotides (AONs) to reduce expression of the transactive response DNA binding protein 43 (TARDBP) gene which encodes the protein TAR DNA-binding protein 43 (TDP43). The invention provides methods to treat, prevent or ameliorate the effects of a disease associated with TDP43 proteinopathy by administration of AONs and therapeutic compositions comprising AONs targeted to TARDBP. The invention also relates to AONs that are targeted to nucleic acids encoding STMN2 and are capable of binding to STMN2 pre-mRNA.

### BACKGROUND ART

The following discussion of the background art is intended to facilitate an understanding of the present invention only. The discussion is not an acknowledgement or admission that any of the material referred to is or was part of the common general knowledge as at the priority date of the application.

TDP43 proteinopathy involves the aberrant mislocalisation, phosphorylation, ubiquitination and cleavage of TDP43 in neurons and glial cells. TDP43 proteinopathy occurs across several neurodegenerative diseases. Histological studies have confirmed that TDP43 is present in cytoplasmic aggregates in the neurons of the majority of amyotrophic lateral sclerosis (ALS) patients, including those with pathogenic variations in the TARDBP gene as well as in sporadic cases, and in those with C90RF72 hexanucleotide repeat expansions (Giordana et. al. Brain Pathology, 2010; Takeuchi et al. Acta Neuropathologica Communications 2016; Schipper et al. Neuropathology and Applied Neurobiology, 2016). The mislocalisation and aggregation of TDP43 in ubiquitin-positive cytoplasmic neuronal inclusions in the brain and spinal cord is now considered a pathological hallmark of ALS. TDP43 proteinopathy is also seen in the most common subtype of frontotemporal lobar degeneration (FTLD) (Gao et al. Journal of neurochemistry, 2018), also known as frontotemporal degeneration or frontotemporal dementia (FTD), and in 30 to 70% of Alzheimer's disease (AD) cases (Josephs et al. Acta Neuropathologica, 2016). TDP43 pathology is also a feature in several other neurodegenerative or neuromuscular diseases including Perry syndrome (PS) (Mishima, T., et al., Journal of neuropathology and experimental neurology, 2017. 76(8): p. 676-682)., chronic traumatic encephalopathy (CTE) (McKee, A. C., et al., Acta neuropathologica, 2015. 131(1): p. 75-86), neurodegeneration with brain iron accumulation type 1 (NBIAT1) (Haraguchi, T., et al., Neuropathology, 2011. 31(5): p. 531-539), anti-IgLON5 syndrome (AIS) (Cagnin, A., et al., Journal of Alzheimer's disease, 2017. 59(1): p. 13-20), neuronal ceroid lipofuscinosis (NCL) (Götzl, J. K., et al., Acta neuropathologica, 2014. 127(6): p. 845-860), lewy body dementia (LBD) (Arai, T., et al., Acta neuropathologica, 2009. 117(2): p. 125-136.), inclusion body myopathy (IBMY) (Weihl, C. C., et al., Journal of Neurology, Neurosurgery & Psychiatry, 2008. 79(10): p. 1186-1189), inclusion body myositis (IBM) (Huntley, M. L., et al., Laboratory Investigation, 2019. 99(7): p. 1041-1048) and in several other very rare diseases.

The TARDBP gene has been implicated in ALS. ALS is a fatal degenerative disease that affects motor neurons. ALS typically occurs in mid-life and presents as a relentlessly progressive muscle atrophy and weakness, with the effects on respiratory muscles limiting survival to 2 to 4 years after disease onset in most cases (Chio et al. World Federation of Neurology Research Group on Motor Neuron Diseases, 2009). ALS is the most common adult motor neuron disease with an incidence of 2 per 100,000 and prevalence of 5.4 per 100,000 individuals (Chio et al. Neuroepidemiology, 2013). Current treatment options are based on symptom management and respiratory support with the only approved medications prolonging survival for just a few months (Cetin H. et al, Neuroepidemiology, 2015. 44: p. 6-1) or providing only modest benefits in some patients (Sawada, Expert Opinion on Pharmacotherapy, 2017. 18(7): p. 735-738). Effective treatments that slow or pause disease progression are lacking.

Although usually concentrated in the nucleus, TDP43 contains both a nuclear localization signal and a nuclear export signal and shuttles back and forth between the nucleus and cytoplasm (Ayala et al. Journal of Cell Science, 2008. 121(22): p. 3778-3785). Expression of TDP43 is autoregulated through a feedback mechanism in which the protein binds to a region within the 3′UTR of its own pre-mRNA when in nuclear excess. This triggers the use of alternative polyadenylation signals and splicing events that result in mRNA transcripts that are degraded rather than translated (Koyama et al., Nucleic acids research, 2016. 44(12): p. 5820-5836; Avendarño-Vázquez, S. E., et al., Genes & Development, 2012. 26(15): p. 1679-1684; D'Alton et al., RNA, 2015. 21(8): p. 1419-1432).

The cytoplasmic mislocalisation of TDP43 in ALS leads to its runaway upregulation. This results in increased levels and the formation of potentially toxic aggregations of TDP43 in the cytoplasm, whilst TDP43 levels in the nucleus remain depleted (Koyama et al., Nucleic Acid Res. 2016. July; 44(12): p 5820-36). TDP43 overexpression rodent models have consistently found that overexpression of both wild-type and mutant TDP43 can cause a neurodegenerative phenotype (Ash et al., Human Molecular Genetics, 2010. 19(16): p. 3206-3218; Wils et al., PNAS USA, 2010. 107(8): p. 3858-3863; Kabashi, et al., Human Molecular Genetics, 2010. 19(4): p. 671-683; Stallings et al., Neurobiology of Disease, 2010. 40(2): p. 404-414; Xu et al., Molecular Neurodegeneration, 2011. 6(1): p. 73; Liachko, et al., The Journal of Neuroscience, 2010. 30(48): p. 16208-16219).

TDP43 functions as a regulator of gene expression and is involved in several RNA processing steps with roles in pre-mRNA splicing, regulation of mRNA stability, mRNA transport, translation and the regulation of non-coding RNAs (Ratti, A. and E. Buratti, Journal of Neurochemistry, 2016. 138(S1): p. 95-111; Buratti, E. and F. E. Baralle, RNA Biology, 2010. 7(4): p. 420-429; Tollervey et al., Nature Neuroscience, 2011. 14(4): p. 452-458). It also plays a role in stress granule dynamics. Stress granules facilitate cell survival by the translational arrest of non-essential transcripts and pro-apoptotic proteins when under stress (Protter, D. S. W. and R. Parker, Trends in Cell Biology, 2016. 26(9): p. 668-679).

TDP43 has functions in both the nucleus and cytoplasm but predominantly resides in the nucleus. Through its roles in splicing and transcription, TDP43 is involved in the regulation of many other genes and its nuclear depletion is likely to lead to various downstream effects in the cell.

Increased cytoplasmic TDP43 effects global mRNA translation, stress granule dynamics, mitochondrial functioning and other cellular pathways. TDP43 is involved in regulating translation with its cytoplasmic increase shown to lead to a global reduction in protein synthesis (Russo et al., Human Molecular Genetics, 2017. 26(8): p. 1407-1418). TDP43 is also involved in stress granule formation and maintenance (Khalfallah, Y., et al., Scientific Reports, 2018. 8(1): p. 1-13). Proteins with prion-like domains such as TDP43 are thought to be vital for the reversible assembly of stress granules due to their capacity for forming multiple transient weak interactions (Harrison, A. F. and J. Shorter, The Biochemical Journal, 2017. 474(8): p. 1417-1438). Increased cytoplasmic TDP43 may interfere with stress granule dynamics leading to their dysfunction. TDP43 overexpression also effects mitochondrial function in several ways (Wang, W., et al., Nature Medicine, 2016. 22(8): p. 869-87827; Prasad, A., et al., Frontiers in Molecular Neuroscience, 2019. 12(25)). The accumulation of cytoplasmic TDP43 may also contribute to disease progression through a prion-like propagation mechanism (Nonaka, T., et al., Cell Reports, 2013. 4(1): p. 124-134).

The depletion of nuclear TDP43 is an issue, and therapeutic strategies that ameliorate the worst effects of TDP43 nuclear depletion are being explored. However, increased cytoplasmic TDP43 is associated with significant adverse consequences. Therefore, strategies to reduce cytoplasmic TDP43 may be effective for treating diseases associated with TDP43 proteinopathy.

AON mediated TDP43 downregulation may reduce the impact of cytoplasmic TDP43 overexpression on these processes. WO2019/013141 describes a number of AONs directed to TARDBP. However, none of these AONs have as yet led to an effective commercially available treatment for diseases associated with TDP43 proteinopathy.

Through its roles in splicing and transcription, TDP43 is involved in the regulation of many other genes and its nuclear depletion is likely to lead to various downstream effects in the cell. Nuclear TDP43 depletion leads to the differential expression or splicing of hundreds of RNAs (Klim et al., Nature Neuroscience, 2019. 22(2): p. 167-179) [1][1]. Recent studies have shown that TDP43 proteinopathy in ALS alters the expression of neuronal protein stathmin-2 and that this may have a direct functional link to enhanced neuronal vulnerability.

Stathmin-2 is a neuronal phosphoprotein encoded by the STMN2 gene. It is a membrane-associated protein that is localised to the Golgi as well as distributed in vesicles to the perinuclear cytoplasm, axons, and growth cones of neurons (Chauvin, S. and A. Sobel, Neuronal stathmins: A family of phosphoproteins cooperating for neuronal development, plasticity and regeneration. Progress in Neurobiology, 2015. 126: p. 1-18). Stathmin-2 is highly expressed in the nervous system and is upregulated during neuronal differentiation, plasticity and regeneration (Chauvin and Sobel 2015).

Stathmin-2 stimulates neurite outgrowth through modulation of microtubule dynamics within growth cones (Morii, H., Y. Shiraishi-Yamaguchi, and N. Mori, SCG10, a microtubule destabilizing factor, stimulates the neurite outgrowth by modulating microtubule dynamics in rat hippocampal primary cultured neurons. Journal of Neurobiology, 2006. 66(10): p. 1101-1114). Microtubules are polymers of tubulin that make up part of the cytoskeleton, providing structure and shape to a cell. Microtubules are dynamic structures that undergo phases of assembly and depolymerisation that is dependent on the amount of free tubulin in the environment (Chauvin and Sobel 2015). Stathmins, including stathmin-2, regulate the amount of tubulin available via its sequestration and release. Phosphorylation of stathmin-2 is a negative regulator of tubulin sequestration, promoting tubular release and polymerisation (Poulain and Sobel, Molecular and Cellular Neuroscience, 2007. 34(2): p. 137-146). The tight regulation of stathmin-2 is required for proper neuritogenesis. . Stathmin-2 depletion induces prolonged growth cone pauses and increased surface area (Poulain and Sobel, Molecular and Cellular Neuroscience, 2007. 34(2): p. 137-146). Moderate levels of stathmin-2 stimulate neurite outgrowth while overexpression leads to neurite retraction due to excess microtubule disassembly (Morii et al Neurobiology, 2006. 66(10): p. 1101-1114). Stathmins play important roles in regenerative processes in the central and peripheral nervous system (Chauvin and Sobel 2015)[2] and are upregulated during peripheral nerve regeneration and after brain trauma (Voria et al., Experimental Neurology, 2006. 197(1): p. 258-267; Shin et al, Experimental neurology, 2014. 252: p. 1-11). Stathmin-2 also plays a role in other neuronal functions including intracellular trafficking and neuroendocrine secretion (Mahapatra et al Biochemistry, 2008. 47(27): p. 7167-7178).

In neurons, stathmin-2 levels are regulated by TDP43. TDP43 is able to bind to a site within the first intron of STMN2 pre-mRNA; this suppresses the inclusion of a cryptic exon into the mature mRNA transcript allowing the full STMN2 mRNA and the protein to be expressed. When TDP43 levels are lowered, its binding to STMN2 pre-mRNA is decreased, leaving the cryptic exon exposed and leading to its inclusion in the mature mRNA transcript. The cryptic exon contains a premature termination codon as well as a premature polyadenylation site, whose utilisation leads to a truncated and non-functional mRNA resulting in decreased stathmin-2 expression (Melamed et al., Nature neuroscience, 2019. 22(2): p. 180-190).

Studies in ALS patient iPSC derived motor neurons depleted of TDP43 report reduced stathmin-2 expression that leads to reduced axonal outgrowth and regeneration that can be restored by increasing its expression (Melamed et al., Nature neuroscience, 2019. 22(2): p. 180-190). Reduced stathmin-2 expression and increased expression of STMN2 transcripts containing the cryptic exon have been confirmed in vivo in motor neurons from tissue samples taken from ALS patients (Klim et al., Nature Neuroscience, 2019. 22(2): p. 167-179; Melamed, Z.e., et al., Nature neuroscience, 2019. 22(2): p. 180-190). A therapeutic strategy that combines AONs designed to reduce TDP43 expression and prevent the subsequent downregulation of stathmin-2 may reduce the impact of cytoplasmic TDP43 overexpression whilst protecting against the neuronal vulnerability caused by stathmin-2 depletion.

Antisense technology is emerging as an effective means for reducing the expression of specific gene products and may therefore prove to be uniquely useful in a number of therapeutic, diagnostic, and research applications for the modulation of TARDBP expression. Antisense technology can affect gene expression at a variety of different levels (transcription, splicing, stability, translation). However, the challenge with antisense technology is that it remains difficult to identify specific AONs that have the desired effect in vivo.

Therefore, notwithstanding a significant amount of research, there still remains a need to develop and identify effective treatments for neurological conditions such as ALS. There is still no clear path to this treatment.

It is in the light of this background that the present invention has been developed. Particularly, the present invention seeks to provide a means for ameliorating TDP43 proteinopathy in diseases associated with TDP43 proteinopathy.

### SUMMARY OF THE INVENTION

The present invention is directed to compounds, particularly AONs, which are targeted to a nucleic acid encoding TARDBP. Embodiments of the present invention relate to AONs that are capable of binding to TARDBP pre-mRNA. The present invention is also directed to compounds, particularly AONs, which are targeted to a nucleic acid encoding STMN2. Embodiments of the present invention relate to AONs that are capable of binding to STMN2 pre-mRNA.

Broadly, according to the first aspect of the invention, there is provided an antisense oligonucleotide targeted to a nucleic acid molecule encoding TARDBP pre-mRNA, wherein the antisense oligonucleotide has a nucleobase sequence that is: (a) selected from the list consisting of: SEQ ID NO: 1 to SEQ ID NO: 25, SEQ ID NO: 38 to 58 or a variant thereof; or (b) complementary to at least 1 or more contiguous nucleobases in a target TARDBP pre-mRNA to which SEQ ID NO: 1 to SEQ ID NO: 25, SEQ ID NO: 38 to 58 also binds or a variant thereof, wherein the antisense oligonucleotide inhibits the expression of the TARDBP gene and wherein the antisense oligonucleotide is substantially isolated or purified.

In one embodiment, the antisense oligonucleotide inhibits the expression of TDP43.

In a further embodiment, the antisense oligonucleotide binds to exon 3 on TARDBP.

In a further embodiment, the antisense oligonucleotide induces alternative splicing of TARDBP pre-mRNA through exon skipping.

In a further embodiment, the exon is exon 3.

In a further embodiment, the antisense oligonucleotide is a phosphorodiamidate morpholino oligomer.

In a further embodiment, the antisense oligonucleotide is a peptide-phosphorodiamidate morpholino oligomer conjugate.

In a further embodiment, the antisense oligonucleotide is selected from the list consisting of: SEQ ID NO: 12, 16, 24 and 25. Preferably, the antisense oligonucleotide is SEQ ID NO: 16, 25.

In a further embodiment, the antisense oligonucleotide is a phosphorodiamidate morpholino oligomer.

In a second aspect of the invention, there is provided an antisense oligonucleotide targeted to a nucleic acid molecule encoding STMN2 pre-mRNA, wherein the antisense oligonucleotide has a nucleobase sequence that is: selected from the list consisting of: (a) SEQ ID NO: 29 to SEQ ID NO: 37, SEQ ID NO: 62 to 66 or a variant thereof; or (b) complementary to at least 1 or more contiguous nucleobases in a target STMN2 pre-mRNA to which SEQ ID NO: 29 to SEQ ID NO: 37, SEQ ID NO: 62 to 66 also binds or a variant thereof, wherein the antisense oligonucleotide prevents the downregulation of and/or increases expression of the STMN2 gene and wherein the antisense oligonucleotide is substantially isolated or purified.

In a further embodiment, the antisense oligonucleotide prevents or reduces the downregulation of or increases the expression of stathmin-2.

In a further embodiment, the antisense oligonucleotide binds to the cryptic exon in intron 1 of human STMN2.

In a further embodiment, the antisense oligonucleotide leads to the cryptic exon being excluded from the mature mRNA transcript.

In a further embodiment, the antisense oligonucleotide is a phosphorodiamidate morpholino oligomer.

In a further embodiment, the antisense oligonucleotide is a peptide-phosphorodiamidate morpholino oligomer conjugate.

In a further embodiment, the antisense oligonucleotide is selected from the list consisting of: SEQ ID NO: 36 and 37.

In a further embodiment, the antisense oligonucleotide is a phosphorodiamidate morpholino oligomer.

In another aspect of the invention, there is provided a method of inducing alternative splicing of TARDBP pre-mRNA, the method comprising the steps of: (a) providing one or more of the antisense oligonucleotides according to the first aspect of this invention; and (b) allowing the oligomer(s) to bind to a target nucleic acid site.

In another aspect of the invention, there is provided a method of inducing alternative splicing of STMN2 pre-mRNA, the method comprising the steps of: (a) providing one or more of the antisense oligonucleotides to the second aspect of this invention; and (b) allowing the oligomer(s) to bind to a target nucleic acid site.

In another aspect of the invention, there is provided a composition to treat, prevent or ameliorate the effects of a disease associated with TDP43 proteinopathy, the composition comprising: (a) one or more antisense oligonucleotides according to the first aspect of this invention; and (b) one or more therapeutically acceptable carriers and/or diluents.

In a further embodiment, the composition further comprises one or more antisense oligonucleotides according to the second aspect of this invention.

In another aspect of the invention, there is provided a pharmaceutical composition to treat, prevent or ameliorate the effects of a disease associated with TDP43 proteinopathy, the composition comprising: (a) one or more antisense oligonucleotides according to the first aspect of this invention; and (b) one or more pharmaceutically acceptable carriers and/or diluents.

In a further embodiment, the disease associated with TDP43 proteinopathy is selected from the group consisting of: ALS, AD, FTLD, PS, CTE, NBIAT1, AIS, NCL, LBD, IBMY and IBM.

In a further embodiment, the pharmaceutical composition further comprises one or more antisense oligonucleotides according to the second aspect of this invention.

In another aspect of the invention, there is provided a method of treating, preventing or ameliorating the effects of a disease associated with TDP43 proteinopathy, the method comprising the step of administering to the subject an effective amount of the pharmaceutical composition of the invention.

In a further embodiment, the disease associated with TDP43 proteinopathy is selected from the group consisting of: ALS, AD, FTLD, PS, CTE, NBIAT1, AIS, NCL, LBD, IBMY and IBM.

In a further embodiment, the disease associated with TDP43 proteinopathy is selected from the group consisting of: ALS, AD and FTLD, PS, IBMY, IBM, CTE, LBD, and NCL.

In a further embodiment, the disease associated with TDP43 proteinopathy is selected from the group consisting of: ALS, AD and FTLD, PS, IBMY and IBM.

In a further embodiment, the disease associated with TDP43 proteinopathy is selected from the group consisting of: ALS, AD and FTLD.

In another aspect of the invention, there is provided a method for treating, preventing or ameliorating the effects of a disease associated with TDP43 proteinopathy in patients identified by a biomarker, the method comprising the step of: (a) testing a subject for the presence of a biomarker associated with a disease associated with TDP43 proteinopathy patients likely to respond to TDP43 suppression; and (b) if the subject is found to express the biomarker, administering to the subject an effective amount of the pharmaceutical composition of the invention.

In a further embodiment, the disease associated with TDP43 proteinopathy is selected from the group consisting of: ALS, AD, FTLD, PS, CTE, NBIAT1, AIS, NCL, LBD, IBMY and IBM.

In a further embodiment, the biomarker is a truncated STMN2 transcript.

In another aspect of the invention, there is provided a method of reducing the expression of TDP43 in a subject and/or reducing the overexpression of TDP43 caused by auto regulation in a subject, the method comprising the step of administering to the subject an effective amount of the pharmaceutical composition of the invention.

In another aspect of the invention, there is provided a method of preventing or reducing the downregulation of the STMN2 gene to maintain normal physiological levels of stathmin-2 and/or increase stathmin-2 expression where it has been reduced in the subject, the method comprising the step of administering to the subject an effective amount of a pharmaceutical composition comprising: (a) one or more antisense oligonucleotides according to the second aspect of this invention; and (b) one or more pharmaceutically acceptable carriers and/or diluents.

In another aspect of the invention, there is provided a method of: (1) reducing the expression of TDP43 in a subject; and/or (2) reducing the over expression of TDP43 caused by auto regulation in a subject and preventing or reducing the downregulation of the STMN2 gene to maintain normal physiological levels or increased levels of stathmin-2 in the subject, the method comprising the step of administering to the subject an effective amount of a pharmaceutical composition comprising: one or more antisense oligonucleotides according to the first aspect of the invention; one or more antisense oligonucleotides according to the second aspect of the invention; and one or more pharmaceutically acceptable carriers and/or diluents.

In another aspect of the invention, there is provided a method of: (1) reducing the expression of TDP43 in a subject; and/or (2) reducing the over expression of TDP43 caused by auto regulation in a subject and preventing or reducing the downregulation of the STMN2 gene to maintain normal physiological levels of or increased levels of stathmin-2 in the subject, the method comprising the step of administering to the subject an effective amount of: (a) a pharmaceutical composition comprising one or more antisense oligonucleotides according to the first aspect of the invention, and one or more pharmaceutically acceptable carriers and/or diluents; and (b) a second pharmaceutical composition comprising one or more antisense oligonucleotides according to a second aspect of the invention, and one or more pharmaceutically acceptable carriers and/or diluents, wherein the two pharmaceutical compositions are administered to the subject concurrently or sequentially.

In another aspect of the invention, there is provided an expression vector comprising one or more antisense oligonucleotides according to the first aspect of this invention.

In a preferred embodiment, the expression vector comprises one or more antisense oligonucleotides of the second aspect of the invention.

In another aspect of the invention, there is provided an expression vector comprising one or more antisense oligonucleotides according to the second aspect of this invention.

In another aspect of the invention, there is provided a cell comprising the antisense oligonucleotide according to the first aspect of the invention.

In a preferred embodiment, the cell comprises one or more antisense oligonucleotides according to the second aspect of this invention.

In another aspect of the invention, there is provided a cell comprising the antisense oligonucleotide according to second aspect of this invention.

In another aspect of the invention, there is provided the use of antisense oligonucleotides according to the first aspect of this invention, for the manufacture of a medicament to treat, prevent or ameliorate the effects of a disease associated with TDP43 proteinopathy.

In another aspect of the invention, there is provided the use of antisense oligonucleotides according to the first aspect of this invention, to treat, prevent or ameliorate the effects of a disease associated with TDP43 proteinopathy.

In a preferred embodiment, the disease associated with TDP43 proteinopathy is selected from the group consisting of: ALS, AD, FTLD, PS, CTE, NBIAT1, AIS, NCL, LBD, IBMY and IBM.

In a preferred embodiment, the use comprises the use of one or more of the antisense oligonucleotides according to the second aspect of the invention.

In another aspect of the invention, there is provided a kit to treat, prevent or ameliorate the effects of a disease associated with TDP43 proteinopathy in a subject, wherein the kit comprises at least an antisense oligonucleotide according to the first aspect of the invention, packaged in a suitable container, together with instructions for its use.

In a preferred embodiment, the disease associated with TDP43 proteinopathy is selected from the group consisting of: ALS, AD, FTLD, PS, CTE, NBIAT1, AIS, NCL, LBD, IBMY and IBM.

In a preferred embodiment, the kit further comprises an antisense oligonucleotide according to a second aspect of the invention.

The invention extends, according to a still further aspect thereof, to cDNA or cloned copies of the AON sequences of the invention, as well as to vectors containing one or more of the AON sequences of the invention. The invention extends further to cells containing such sequences and/or vectors.

Further features of the present invention are more fully described in the following description of several non-limiting embodiments thereof. This description is included solely for the purposes of exemplifying the present invention. It should not be understood as a restriction on the broad summary, disclosure or description of the invention as set out above.

### BRIEF DESCRIPTION OF DRAWINGS

The following description is provided with reference to the following accompanying drawings.

FIG. 1 shows a schematic of the transcripts of TARDBP that are produced when different polyadenylation sites are utilised.

FIG. 2 shows a schematic of the binding location of each of the exon 2 and 3 targeted AONs on TARDBP.

FIG. 3 shows TARDBP transcript analysis via RT-PCR and agarose gel electrophoresis following transfection with TARDBP exon 2 targeted AOs in human fibroblasts.

FIG. 4 shows TARDBP transcript analysis via RT-PCR and agarose gel electrophoresis following transfection with TARDBP exon 3 targeted AONs in human fibroblasts.

FIG. 5 shows TARDBP transcript analysis via RT-PCR and agarose gel electrophoresis for the investigation into the mechanism of TARDBP transcript knockdown induced by exon 2 targeted AONs SEQ ID NO: 3 and 12.

FIG. 6 shows TARDBP transcript analysis via RT-PCR and agarose gel electrophoresis following transfection of human fibroblasts with AONs designed to block TARDBP polyadenylation site 1 (PA1).

FIG. 7 shows TARDBP transcript analysis and TDP43 protein levels after transfection of human fibroblasts via nucleofection with TARDBP targeted PMOs AON ID 24 and 25 at 150 μM.

FIG. 8 shows results for 3 transfections of human fibroblasts via nucleofection with TARDBP targeted PMO (AON 25) at 100 and 50 μM concentrations. Error bars represent standard error of the mean.

FIG. 9 shows STMN2 transcript analysis via RT-PCR and agarose gel electrophoresis following transfection with TARDBP targeted PMO (AON 25) in human SH-SY5Y cells.

FIG. 10 shows a schematic of the binding location of the STMN2 cryptic exon targeted AONs in intron 1 of STMN2.

FIG. 11 shows STMN2 transcript analysis after transfection of human SH-SH5Y cells via electroporation with TARDBP exon 3 skipping PMO (SEQ ID NO: 25) alone or in combination with the STMN2 cryptic exon targeted 2′ O Methyl-PS AONs (SEQ IDs 29, 30 or 31) or a control oligo (AON ID 28).

FIG. 12 shows STMN2 transcript analysis via RT-PCR and agarose gel electrophoresis following sequence optimisation by shifting the AON sequence 5 bases upstream and downstream of SEQ ID NO: 29 (SEQ IDs 32 and 33) and SEQ ID NO: 30 (SEQ ID 34 and 35). Error bars represent standard error of the mean.

FIG. 13 shows STMN2 transcript analysis after transfection of human SH-SH5Y cells via electroporation with TARDBP exon 3 skipping PMO (SEQ ID NO: 25) or a combination of SEQ ID NO: 25 and STMN2 targeted SEQ IDs 36 or 37.

FIG. 14 shows STMN2 transcript analysis results for 3 transfections of SH-SY5Y cells with TARDBP exon 3 skipping PMO (SEQ ID NO: 25) or a combination of SEQ ID NO: 25 and STMN2 targeted SEQ IDs 36 or 37. Error bars represent standard error of the mean.

FIG. 15 shows representative western blot images and densitometric analysis for TARDBP SEQ ID NO: 25 and STMN2 SEQ IDs 36 or 37 co-treated cells. Error bars represent standard error of the mean.

FIG. 16 shows STMN2 transcript analysis after transfection of human SH-SH5Y cells via electroporation with SEQ ID NO: 37. Error bars represent standard error of the mean.

FIG. 17 shows results of the stathmin-2 expression represented by Western blot protein analysis for SEQ ID NO: 37 only treated cells.

### DETAILED DESCRIPTION

The present invention provides a prophylactic or therapeutic method for ameliorating or slowing the further progress of symptoms of diseases associated with TDP43 proteinopathy (including ALS, AD, FTLD, PS, CTE, NBIAT1, AIS, NCL, LBD, IBMY and IBM) using AON therapy. More specifically, the invention provides isolated or purified AONs targeted to a nucleic acid molecule encoding TARDBP pre-mRNA, wherein the AON has a nucleobase sequence that is:
a. selected from the list comprising SEQ ID NO: 1 to SEQ ID NO: 25; SEQ ID NO: 38 to SEQ ID NO: 58 inclusive or variants thereof, or
b. a sequence that is complementary to at least 1 or more contiguous nucleobases in a target TARDBP pre-mRNA to which SEQ ID NO: 1 to SEQ ID NO: 25; SEQ ID NO: 38 to SEQ ID NO: 58 inclusive or variants thereof, also bind, and
c. wherein the AON inhibits the expression of human TARDBP.

The invention also provides isolated or purified antisense oligonucleotides that target to a nucleic acid molecule encoding STMN2 pre-mRNA, wherein the AON has a nucleobase sequence that is:
a. selected from the list comprising SEQ ID NO: 29 to SEQ ID NO: 37, SEQ ID NO: 62 to 66 inclusive, or
b. a sequence that is complementary to at least 1 or more contiguous nucleobases in a target STMN2 pre-nRNA to which SEQ ID NO: 29 to SEQ ID NO: 37, SEQ ID NO: 62 to 66 inclusive also bind, and
wherein the AON maintains normal physiological levels of and/or increases expression of stathmin-2 when it is reduced.

For convenience, the following sections generally outline the various meanings of the terms used herein. Following this discussion, general aspects regarding compositions, use of medicaments and methods of the invention are discussed, followed by specific examples demonstrating the properties of various embodiments of the invention and how they can be employed.

### 1. Definitions

The meaning of certain terms and phrases used in the specification, examples, and appended claims, are provided below. If there is an apparent discrepancy between the usage of a term in the art and its definition provided herein, the definition provided within the specification shall prevail.

Those skilled in the art will appreciate that the invention described herein is susceptible to variations and modifications other than those specifically described. The invention includes all such variations and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness. None of the cited material or the information contained in that material should, however, be understood to be common general knowledge.

Manufacturer's instructions, descriptions, product specifications, and product sheets for any products mentioned herein or in any document incorporated by reference herein, are hereby incorporated herein by reference, and can be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

Other than in the operating examples, or where otherwise indicated, all numbers expressing quantities of ingredients or reaction conditions used herein should be understood as modified in all instances by the term “about.” The term “about” when used in connection with percentages can mean±1%.

The invention described herein may include one or more range of values (e.g., size, concentration etc.). A range of values will be understood to include all values within the range, including the values defining the range, and values adjacent to the range which lead to the same or substantially the same outcome as the values immediately adjacent to that value which defines the boundary to the range. For example, a person skilled in the field will understand that a 10% variation in upper or lower limits of a range can be totally appropriate and is encompassed by the invention. More particularly, the variation in upper or lower limits of a range will be 5% or as is commonly recognised in the art, whichever is greater.

In this application, the use of the singular also includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

Throughout this specification, unless the context requires otherwise, the word “comprise” or variations such as “comprises” or “comprising”, will be understood to imply the inclusion of a stated integer or group of integers but not the exclusion of any other integer or group of integers.

As used herein, the term “administer” refers to the placement of a composition into a subject by a method or route which results in at least partial localization of the composition at its desired site of action such that desired effect is produced. A compound or composition described herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

Features of the invention will now be discussed with reference to the following non-limiting description and examples.

### 2. Embodiments

Embodiments of the present invention relate generally to improved antisense compounds, and methods or use thereof, which are specifically designed to supress the expression of TARDBP. Mislocalisation of TARDBP in the cytoplasm has been implicated in disease associated with TDP43 proteinopathy including ALS, FTLD and AD.

Without being bound by theory, the present invention is based on the understanding that suppressing the expression of TDP43 in patients suffering from a disease associated with TDP43 proteinopathy may have the effect of slowing progressing of symptoms and/or improving survival of these patients. This is because TDP43 proteinopathy, including cytoplasmic mislocalisation of TDP43 which leads to runaway upregulation of TDP43, is associated with a number of neurological conditions including ALS, FTLD and AD. Therefore, the suppression of the TARDBP gene (which encodes TDP43) is hypothesised to result in slowing progressing of symptoms and/or improving survival of patients suffering from disease associated with TDP43 proteinopathy including ALS, FTLD and AD. The patients that can benefit from this therapy may have mutations or misfolding in the TARDBP gene. However, patients that do not exhibit TARDBP mutations or misfolding may also respond to treatment supressing the TARDBP gene.

Embodiments of the present invention also relate generally to improved antisense compounds, and methods or use thereof, which are specifically designed to prevent the subsequent downregulation of stathmin-2. Stathmin-2 plays an important role in regenerative processes in the central and peripheral nervous system. Reduced stathmin-2 expression has been confirmed in vivo in motor neurons from tissue samples taken from ALS patients. Without being bound by theory, the present invention is also based on the understanding that a therapeutic strategy that combines AONs designed to reduce TDP43 expression with AONs designed to prevent the subsequent downregulation of stathmin-2, may reduce the impact of cytoplasmic TDP43 overexpression whilst protecting against the neuronal vulnerability caused by stathmin-2 depletion. Normal physiological levels of the stathmin-2 protein may be maintained by targeting AONs to STMN2 transcripts.

### A. Antisense Oligonucleotides

This invention provides one or more isolated or purified AONs that target a nucleic acid molecule encoding TARDBP pre-mRNA, wherein the AON has a nucleobase sequence selected from the list comprising SEQ ID NO: 1 to SEQ ID NO: 25, SEQ ID NO: 38 to 58 inclusive (as set out in Tables 1 and 2, below) and wherein the AON inhibits the expression of human TDP43. Preferably, the AON is a phosphorodiamidate morpholino oligomer.

More generally, the invention provides isolated or purified antisense oligonucleotides that target to a nucleic acid molecule encoding TARDBP pre-mRNA, wherein the AON has a nucleobase sequence that is:
a. selected from the list comprising SEQ ID NO: 1 to SEQ ID NO: 25, SEQ ID NO: 38 to 58 inclusive, or
b. a sequence that is complementary to at least 1 or more contiguous nucleobases in a target TARDBP pre-mRNA to which SEQ ID NO: 1 to SEQ ID NO: 25, SEQ ID NO: 38 to 58 inclusive also bind, and
c. wherein the AON inhibits the expression of human TARDBP.

This invention also provides one or more isolated or purified AONs that target a nucleic acid molecule encoding STMN2 pre-mRNA, wherein the AON has a nucleobase sequence selected from the list comprising SEQ ID NO: 29 to SEQ ID NO: 37, SEQ ID NO: 62 to 66 inclusive (as set out in Tables 1 and 2, below) and wherein these AONs maintain normal physiological levels of or reduce downregulation of stathmin-2. More generally, the invention also provides isolated or purified antisense oligonucleotides that target to a nucleic acid molecule encoding STMN2 pre-mRNA, wherein the AON has a nucleobase sequence that is:
a. selected from the list comprising SEQ ID NO: 29 to SEQ ID NO: 37, SEQ ID NO: 62 to 66 inclusive, or
b. a sequence that is complementary to at least 1 or more contiguous nucleobases in a target STMN2 pre-mRNA to which SEQ ID NO: 29 to SEQ ID NO: 37, SEQ ID NO: 62 to 66 inclusive also bind, and
c. wherein these AONs maintain normal physiological levels of or reduce downregulation of stathmin-2.

Preferably, the AON is a phosphorodiamidate morpholino oligomer.

TABLE 1

| SEQ ID | | |
|---|---|---|
| NO | Co-ordinates | Sequence (5′ to 3′) |
| 1 | TARDBP H2A(−22+03) | UUCCUAAAGAGAAAAGAGAUAUGAA |
| 2 | TARDBP H2A(+64+83) | CCAUCGUCUUCCGAUGGUAU |
| 3 | TARDBP H2A(+85+110) | GCUGUAACCGUGGAGAGCAGCACCG |
| 4 | TARDBP H2A | ACACGCCCCUGGAAACUGGGCUGUA |
| | (+105+129) | |
| 5 | TARDBP H2A | GGGCAUGCAGAAUUCCUUCUAC |
| | (+181+202) | |
| 6 | TARDBP H2D(+23-02) | ACCUUUUGGAUAGUUGACAACAUAC |
| 7 | TARDBP H3A(−05+20) | AUCCAUUUUUCUUUUGUUAUCUAAA |
| 8 | TARDBP H3A(+24+48) | CUUUCACUGCUGAUGAAGCAUCUGU |
| 9 | TARDBP H3A(+64+88) | AACACUAUUAAAUCGGAUGUUUUCU |
| 10 | TARDBP H3A(+97+122) | CAGGUCCUGUUCGGUUGUUUUCCAU |
| 11 | TARDBP H3D(+20−05) | UUUACCUGCACCAUAAGAACUUCUC |
| 12 | TARDBP H2A(+80+105) | AACCGUGGAGAGCAGCACCGUCCCA |
| 13 | TARDBP H2A(+90+115) | ACUGGGCUGUAACCGUGGAGAGCAG |
| 14 | TARDBP H2A(+95+120) | UGGAAACUGGGCUGUAACCGUGGAG |
| 15 | TARDBP H3A(+19+43) | ACUGCUGAUGAAGCAUCUGUCUCAU |
| 16 | TARDBP H3A(+29+53) | UUUCACUUUCACUGCUGAUGAAGCA |
| 17 | TARDBP H3A(+59+83) | UAUUAAAUCGGAUGUUUUCUGGACU |
| 18 | TARDBP H3A(+69+93) | GACCCAACACUAUUAAAUCGGAUGU |
| 19 | TARDBP H3A(+93+118) | CCUGUUCGGUUGUUUUCCAUGGGAG |
| 20 | TARDBP H3A | UCUUUCAGGUCCUGUUCGGUUGUUU |
| | (+103+128) | |
| 21 | TARDBP H6A | CAGUUUUAUUUAAGAAUUUC |
| | (+1891+1910) | |
| 22 | TARDBP H6A | GUUUUAUUUAAGAAUUUCCAACAAUGAC |
| | (+1881+1908) | |
| 23 | TARDBP H6A | GACACAUAUUAUUUAAAUCAGUUUUAUUUA |
| | (+1899+1928) | |
| 24 | TARDBP H2A | AACCGTGGAGAGCAGCACCGTCCCA |
| | (+80+105) | |
| 25 | TARDBP H3A | TTTCACTTTCACTGCTGATGAAGCA |
| | (+29+53) | |
| 26 | Ctrl AON 1 | CACCUUCCUUCUUUUUGAUU |
| 27 | Ctrl AON 2 | GGAUGUCCUGAGUCUAGACCCUCCG |
| 28 | Ctrl AON 3 | CCTCTTACCTCAGTTACAATTTATA |
| 29 | STMN2 H2aA | CGAAGGUCUUCUGCCGAGUCCUGCA |
| | (−5+20) | |
| 30 | STMN2 H2aA | CAUUCACAUUCAUUUCUUCUUAGGC |
| | (+100+124) | |
| 31 | STMN2 H2aA | UUUGUUUUAAUUUCUUCAGUAUUGC |
| | (+189+213) | |
| 32 | STMN2 H2aA | GUCUUCUGCCGAGUCCUGCAAUAUG |
| | (−10+15) | |
| 33 | STMN2 H2aA | UCUCUCGAAGGUCUUCUGCCGAGUC |
| | (+1+25) | |
| 34 | STMN2 H2aA | ACAUUCAUUUCUUCUUAGGCAGGCU |
| | (+95+119) | |
| 35 | STMN2 H2aA | AGCCGCAUUCACAUUCAUUUCUUCU |
| | (+105+129) | |
| 36 | STMN2 H2aA | TCTCTCGAAGGTCTTCTGCCGAGTC |
| | (+1+25) | |
| 37 | STMN2 H2aA | AGCCGCATTCACATTCATTTCTTCT |
| | (+105+129) | |

The reference point (0) set at first base of the 5′ and 3′ splice sites; hence “+” refers to nucleotides binding within the exon and “−” indicates nucleotides binding within the intron.

In any of the AONs of the present invention, the uracil (U) of the sequences provided herein may be replaced by a thymine (T). For example, the AONs of the present invention have sequences as listed in Table 2.

TABLE 2

| SEQ | | |
|---|---|---|
| ID NO: | Co-ordinates | Sequence (5′ to 3′) |
| 38 | TARDBP H2A(−22+03) | TTCCTAAAGAGAAAAGAGATATGAA |
| 39 | TARDBP H2A(+64+83) | CCATCGTCTTCCGATGGTAT |
| 40 | TARDBP H2A(+85+110) | GCTGTAACCGTGGAGAGCAGCACCG |
| 41 | TARDBP H2A | ACACGCCCCTGGAAACTGGGCTGTA |
| | (+105+129) | |
| 42 | TARDBP H2A | GGGCATGCAGAATTCCTTCTAC |
| | (+181+202) | |
| 43 | TARDBP H2D(+23−02) | ACCTTTTGGATAGTTGACAACATAC |
| 44 | TARDBP H3A(−05+20) | ATCCATTTTTCTTTTGTTATCTAAA |
| 45 | TARDBP H3A(+24+48) | CTTTCACTGCTGATGAAGCATCTGT |
| 46 | TARDBP H3A(+64+88) | AACACTATTAAATCGGATGTTTTCT |
| 47 | TARDBP H3A(+97+122) | CAGGTCCTGTTCGGTTGTTTTCCAT |
| 48 | TARDBP H3D(+20−05) | TTTACCTGCACCATAAGAACTTCTC |
| 49 | TARDBP H2A(+90+115) | ACTGGGCTGTAACCGTGGAGAGCAG |
| 50 | TARDBP H2A(+95+120) | TGGAAACTGGGCTGTAACCGTGGAG |
| 51 | TARDBP H3A(+19+43) | ACTGCTGATGAAGCATCTGTCTCAT |
| 52 | TARDBP H3A(+59+83) | TATTAAATCGGATGTTTTCTGGACT |
| 53 | TARDBP H3A(+69+93) | GACCCAACACTATTAAATCGGATGT |
| 54 | TARDBP H3A(+93+118) | CCTGTTCGGTTGTTTTCCATGGGAG |
| 55 | TARDBP H3A | TCTTTCAGGTCCTGTTCGGTTGTTT |
| | (+103+128) | |
| 56 | TARDBP H6A | CAGTTTTATTTAAGAATTTC |
| | (+1891+1910) | |
| 57 | TARDBP H6A | GTTTTATTTAAGAATTTCCAACAATGAC |
| | (+1881+1908) | |
| 58 | TARDBP H6A | GACACATATTATTTAAATCAGTTTTATTTA |
| | (+1899+1928) | |
| 59 | Ctrl AON 1 | CACCTTCCTTCTTTTTGATT |
| 60 | Ctrl AON 2 | GGATGTCCTGAGTCTAGACCCTCCG |
| 61 | Ctrl AON 3 | CCTCTTACCTCAGTTACAATTTATA |
| 62 | STMN2 H2aA | CGAAGGTCTTCTGCCGAGTCCTGCA |
| | (−5+20) | |
| 63 | STMN2 H2aA | CATTCACATTCATTTCTTCTTAGGC |
| | (+100+124) | |
| 64 | STMN2 H2aA | TTTGTTTTAATTTCTTCAGTATTGC |
| | (+189+213) | |
| 65 | STMN2 H2aA | GTCTCTGCCGAGTCCTGCAATATG |
| | (−10+15) | |
| 66 | STMN2 H2aA | ACATTCATTTCTTCTTAGGCAGGCT |
| | (+95+119) | |

The reference point (0) set at first base of the 5′ and 3′ splice sites; hence “+” refers to nucleotides binding within the exon and “−” indicates nucleotides binding within the intron.

Further AONs described in the examples of this document include those listed in Table 3.

TABLE 3

| SEQ | | |
|---|---|---|
| ID NO: | Co-ordinates | Sequence (5′ to 3′) |
| 67 | Primer used across | CATTTTGTGGGAGCGAAGCG |
| | TARDBP exons 1 to 6. | |
| 68 | Primer used across | ACGCACCAAAGTTCATCCCA |
| | TARDBP exons 1 to 6. | |
| 69 | Primer. Control gene (TBP) | AGCGCAAGGGTTTCTGGTTT |
| | amplified across exons 2 to | |
| | 3. | |
| 70 | Primer. Control gene (TBP) | GGAGTCATGGGGGAGGGATA |
| | amplified across exons 2 to | |
| | 3. | |
| 71 | Primer used across | TGTACTCCAGCACCATTGGC |
| | STMN2 exons 1 to 3. | |
| 72 | Primer used across | AAAGTTCGTGGGGCTTCTGAG |
| | STMN2 exons 1 to 3. | |
| 73 | Primer used across | TGCTTCAGCCAGACAGTTCA |
| | STMN2 exons 1 to 5. | |
| 74 | Primer used across | GTCAACTGTGCCACAAGCC |
| | STMN2 from exon 1 to a | |
| | position towards the end of | |
| | the cryptic exon sequence. | |

Certain AONs of the invention are designed to complement suitable sequences within the human TARDBP pre-mRNA within exons 2 and 3. In a preferred embodiment, the AONs of the invention are designed to complement suitable sequences within exon 3 of human TARDBP pre-mRNA and induce the skipping of the exon. Most preferably, the AON of the invention is of SEQ ID NO: 16 or SEQ ID NO: 25.

In another preferred embodiment, the AONs of the invention are designed to complement suitable sequences within exon 2. In one embodiment, the AON is of SEQ ID NO: 12 or SEQ ID NO: 24.

Certain AONs of the invention are also designed to complement suitable sequences within the human STMN2 pre-mRNA cryptic exon in intron 1. In a preferred embodiment, the AONs of the invention are designed to complement suitable sequences within cryptic exon in intron 1 of human STMN2 pre-mRNA. Sites were chosen that would inhibit the binding of splicing enhancers as predicted by online splice prediction tools with AONs targeted to three enhancer site hotspots. The reduced binding of splicing enhancers reduces the recognition of the exon by the spliceosome, leading to the cryptic exon being excluded from the mature mRNA transcript. This would lead to increased levels of stathmin-2 being produced as mature STMN2 transcripts that do not contain the cryptic exon are translated.

Preferably, the AON designed to complement suitable sequences within the human STMN2 pre-mRNA is selected from the list of SEQ ID NO: 29 to SEQ ID NO: 37, SEQ ID NO: 62 to 66, or more preferably is selected from SEQ ID NO: 33, 35, 36 and 37.

The terms “antisense oligomer” and “antisense compound” and “antisense oligonucleotide” or “AON” are used interchangeably and refer to a linear sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. The cyclic subunits are based on ribose or another pentose sugar or, in a preferred embodiment, a morpholino group (see description of morpholino oligomers below). The oligomer may have exact or near sequence complementarity to the target sequence; variations in sequence near the termini of an oligomer are generally preferable to variations in the interior. Also contemplated are peptide nucleic acids (PNAs), locked nucleic acids (LNAs), and 2′-O-Methyl oligonucleotides, among other antisense agents known in the art.

The term “oligonucleotide” includes polynucleotides such as deoxyribonucleic acid (DNA) or ribonucleic acid (RNA), with RNA being prepared or obtained by the transcription a DNA template. According to the invention, a nucleic acid may be present as a single-stranded or double-stranded and linear or covalently circularly closed molecule.

By “isolated” it is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally occurring state, e.g., a DNA fragment that is removed from the sequences that are adjacent to the fragment in the genome. The term “isolating” as it relates to cells refers to the purification of cells (e.g., fibroblasts, lymphoblasts) from a source subject (e.g., a subject with a polynucleotide repeat disease). In the context of mRNA or protein, “isolating” refers to the recovery of mRNA or protein from a source, e.g., cells.

An AON can be said to be “directed to” or “targeted against” a target sequence with which it hybridizes. In certain embodiments, the target sequence includes a region including the polyadenylation site and surrounding regions. The target sequence is typically a region including an AUG start codon of an mRNA, a Translation Suppressing Oligomer, or splice site of a pre-processed mRNA, a Splice Suppressing Oligomer (SSO). The target sequence for a splice site may include an mRNA sequence having its 5′ end 1 to about 25 base pairs downstream of a normal splice acceptor junction in a pre-processed mRNA. A preferred target sequence is any region of a pre-processed mRNA that includes a splice site or is contained entirely within an exon coding sequence or spans a splice acceptor or donor site. An oligomer is more generally said to be “targeted against” a biologically relevant target, such as a protein, virus, or bacteria, when it is targeted against the nucleic acid of the target in the manner described above.

As used herein, “sufficient length” or “sufficient sequence complementarity” refers to an AON that is complementary to at least 1, more typically 1-30, contiguous nucleobases in a target TARDBP pre-mRNA (or STMN2 pre-mRNA). In some embodiments, an antisense of sufficient length includes at least 1, 2, 3, 4, 5, 6, 7, 8, 9, 10, 11, 12, 13, 14, or 15 contiguous nucleobases in the target TARDBP pre-mRNA. In other embodiments an antisense of sufficient length includes at least 16, 17, 18, 19, 20, 21, 22, 23, 24, or 25 contiguous nucleobases in the target TARDBP pre-mRNA (or STMN2 pre-mRNA). Preferably, an oligonucleotide of sufficient length is from about 10 to about 50 nucleotides in length, including oligonucleotides of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 and 40 or more nucleotides. In one embodiment, an oligonucleotide of sufficient length is from 10 to about 30 nucleotides in length. In another embodiment, an oligonucleotide of sufficient length is from 15 to about 25 nucleotides in length. In yet another embodiment, an oligonucleotide of sufficient length is from 20 to 30, or 20 to 50, nucleotides in length. In yet another embodiment, an oligonucleotide of sufficient length is from 22 to 28, 25 to 28, 24 to 29 or 25 to 30 nucleotides in length.

In certain embodiments, the AON has sufficient sequence complementarity to a target RNA to block a region of a target RNA (e.g., pre-mRNA) in an effective manner. In some embodiments, such blocking of TARDBP pre-mRNA serves to induce exon skipping. In some embodiments, the target RNA is target pre-mRNA (e.g., TARDBP gene pre-mRNA).

As used herein, the terms “complementary” or “complementarity” are used in reference to polynucleotides {i.e., a sequence of nucleotides) related by the base-pairing rules. For example, the sequence 5′-A-G-T-3′, is complementary to the sequence “′-T-C-A-5′. Complementarity may be “partial”, in which only some of the nucleic acids' bases are matched according to the base pairing rules. Or there may be “complete” or “total” complementarity between the nucleic acids. The degree of complementarity between nucleic acid strands has significant effects on the efficiency and strength of hybridization between nucleic acid strands. As such, a “complement” sequence, as used herein refers to an oligonucleotide sequence have some complementarity to a target RNA or DNA sequence.

For the purpose of the invention, the complement of a nucleotide sequence is the nucleotide sequence which would be capable of forming a double-stranded DNA or RNA molecule with the represented nucleotide sequence, and which can be derived from the represented nucleotide sequence by replacing the nucleotides by their complementary nucleotide according to Chargaff s rules (A< >T; G< >C; A< >U) and reading in the 5′ to 3′ direction, i.e., in opposite direction of the represented nucleotide sequence. This also includes synthetic analogs of DNA/RNA (e.g., 2′ F-ANA oligos).

The term “homology” or “identity” refers to a degree of complementarity. There may be partial homology or complete sequence identity between the oligonucleotide sequence and the complement sequence of the target RNA or DNA. A partially identical sequence is an oligonucleotide that at least partially hybridises to the target RNA or DNA, leading to the formation of partial heteroduplex, and to partial or total degradation of the target RNA or DNA. A completely identical sequence is an oligonucleotide that completely hybrids to the target RNA or DNA, leading to the formation of complete heteroduplex, and to partial or total degradation of the target RNA or DNA.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that cleavage factor binding to the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, (1985), 107-108 or as described in Miyada C. G. and Wallace R. B., (1987), Methods Enzymol. 154, 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-66.

In a preferred embodiment, the AONs of the invention are designed to complement suitable sequences within exon 3 of human TARDBP pre-mRNA and induce the skipping of the exon.

In another preferred embodiment, the AONs of the invention are designed to complement suitable sequences within exon 2.

Preferably, there is provided an AON capable of binding to a selected target site to induce exon skipping in a TARDBP gene transcript or part thereof. In an aspect, the AON induces skipping of exon 2, or 3 in a TARDBP gene. Most preferably, the AON induces skipping of exon 3.

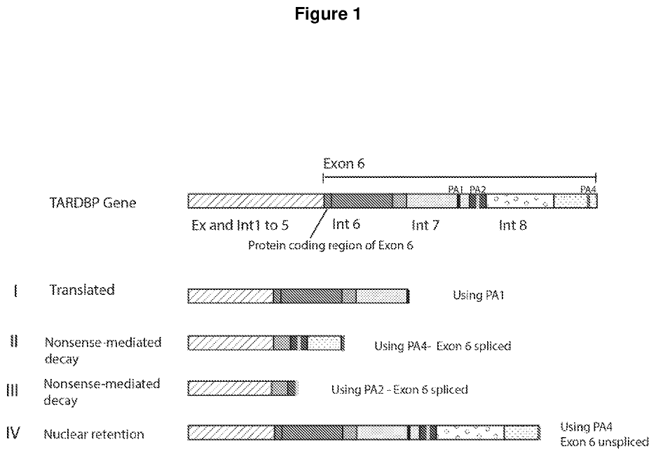

In another embodiment, the AON induces alternative splicing of the TARDBP pre-mRNA by sterically inhibiting the use of PA1 by blocking the polyadenylation signal. Without being bound by theory, the TARDBP transcript is autoregulated via the use of alternative splicing and several polyadenylation signals (PA1, PA2 and PA4) (as described by Koyama et al., 2016). When TDP43 is in nuclear abundance it is able to bind to a location in the 3-UTR of the transcript (Intron 7). This blocks the use of PA1 and triggers the use of PA4 or PA2. When this occurs, the intron 7 acceptor site remains in the transcript allowing intron 7 to be spliced out of the transcript and triggering the subsequent splicing out of introns 6 and 8. The resultant transcripts (II or Ill) are subject to nonsense mediated mRNA decay. A small amount of the unspliced transcript may also remain and is retained in the nucleus. When TDP43 is depleted from the nucleus and is unable to bind to intron 7, PA1 is used. The Int 7 acceptor site does not remain in the transcript as it is downstream of PA1. This transcript (I) is translated to protein. A schematic of the transcripts can be seen in FIG. 1. Therefore, AONs that sterically inhibit the use of PA1 by blocking the polyadenylation signal could lead to a reduction of the translated transcript (1) and an increase in the untranslated transcripts (II, Ill, IV).

The AON is preferably selected from those provided in Table 1 or Table 2. For example, the AON used in the present invention is chosen from the list comprising SEQ ID NO: 12, 16, 24 and 25. Most preferably, the AON is selected from the list comprising SEQ ID NO: 16 or 25.

In another preferred embodiment, the AONs of the invention are designed to complement suitable sequences within cryptic exon in intron 1 of human STMN2 pre-mRNA. Sites were chosen that would inhibit the binding of splicing enhancers as predicted by online splice prediction tools with AONs targeted to three enhancer site hotspots. The reduced binding of splicing enhancers reduces the recognition of the exon by the spliceosome, leading to the cryptic exon being excluded from the mature mRNA transcript. This would lead to increased levels of stathmin-2 being produced as mature STMN2 transcripts that do not contain the cryptic exon are translated.

### B. Methods of Use

The invention further provides a method of inhibiting the expression of TDP43, the method comprising the steps of:
(a)providing one or more of the AONs as described herein and
(b)allowing the oligomer(s) to bind to a target nucleic acid site.

More specifically, the AON may be selected from those set forth in Table 1 or Table 2. The sequences are preferably selected from the group consisting of any one or more of SEQ ID Nos: SEQ ID NO: 1 to SEQ ID NO: 25, SEQ ID NO: 38 to 58, and combinations or cocktails thereof. This includes sequences that can hybridise to such sequences under stringent hybridisation conditions, sequences complementary thereto, sequences containing modified bases, modified backbones, and functional truncations or extensions thereof which possess or modulate RNA processing activity in a TARDBP gene transcript.

Preferably, the AON used in the present invention is chosen from the list comprising SEQ ID NO:12, 16, 24 and 25. Most preferably, the AON is chosen from the list comprising SEQ ID NO: 16 or 25.

In one preferred embodiment, the AONs used in the present method induce alternative splicing of TARDBP pre-mRNA. In an aspect, the AON induces alternative splicing through inducing exon skipping in TARDBP pre-mRNA. Preferably, the AON induces skipping of exon 2 or 3. Most preferably, the AON induces skipping of exon 3. In another embodiment, the AON may reduce the expression of TDP43 by some other mechanism.

As a preferred embodiment, the AON may also be selected from SEQ ID NO: 29 to SEQ ID NO: 37, SEQ ID NO: 62 to 66. These AONs of the invention are designed to complement suitable sequences within the human STMN2 pre-mRNA cryptic exon in intron 1. In a preferred embodiment, certain AONs of the invention are designed to complement suitable sequences within cryptic exon in intron 1 of human STMN2 pre-mRNA. Sites were chosen that would inhibit the binding of splicing enhancers as predicted by online splice prediction tools with AONs targeted to three enhancer site hotspots. The reduced binding of splicing enhancers reduces the recognition of the exon by the spliceosome, leading to the cryptic exon being excluded from the mature mRNA transcript. This would lead to increased levels of stathmin-2 being produced as mature STMN2 transcripts that do not contain the cryptic exon are translated.

A therapeutic strategy that combines: (1) AONs designed to reduce TDP43 expression (SEQ ID NO: 1 to SEQ ID NO: 25, SEQ ID NO: 38 to 58); with (2) AONs designed to prevent or reduce the subsequent downregulation of stathmin-2 (SEQ ID NO: 29 to SEQ ID NO: 37, SEQ ID NO: 62 to 66), will reduce the impact of cytoplasmic TDP43 overexpression whilst protecting against the neuronal vulnerability caused by stathmin-2 depletion. In one aspect, the invention seeks to provide a means for ameliorating TDP43 proteinopathy in a subject suffering from diseases associated with TDP43 proteinopathy whilst maintaining normal physiological levels of stathmin-2.

Preferably, the AON designed to prevent the subsequent downregulation of stathmin-2 is selected from the list of SEQ ID NO: 29 to SEQ ID NO: 37, SEQ ID NO: 62 to 66, or more preferably is selected from SEQ ID NO: 33, 35, 36 and 37.

### Target Sequence and Selective Hybridisation

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs of the present invention may include oligomers that selectively hybridise to the sequences provided in Table 1 or Table 2.

At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the antisense oligomer. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, in some embodiments, the AON sequences of the invention preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the antisense oligomer, the greater the homology required to obtain selective hybridisation. Consequently, where an AON of the invention consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of cleavage modifying activity.

### Physiological Response

In an aspect, the method of the present invention induces a physiological response in a subject. Preferably, the method reduces the expression of TDP43.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound.

By “enhance” or “enhancing,” or “increase” or “increasing,” or “stimulate” or “stimulating,” refers generally to the ability of one or antisense compounds or compositions to produce or cause a greater physiological response (i.e., downstream effects) in a cell or a subject, as compared to the response caused by either no antisense compound or a control compound. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1), e.g., 1.5, 1.6, 1.7, 1.8, etc.) the amount produced by no antisense compound (the absence of an agent) or a control compound.

The terms “decreasing” or “decrease” refer generally to the ability of one or AONs or compositions to produce or cause a reduced physiological response (i.e., downstream effects) in a cell or a subject relative to the response caused by either no AON or a control compound. The term “reduce” or “inhibit” may relate generally to the ability of one or more antisense compounds of the invention to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art and may include reductions in the symptoms or pathology of a TDB-43 related condition. A “decrease” in a response may be statistically significant as compared to the response produced by no antisense compound or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

Relevant physiological or cellular responses (in vivo or in vitro) will be apparent to persons skilled in the art and may include decreases in the amount of TDP43 expression. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7. 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art and may include reductions in the symptoms or pathology of a disease associated with TDP43 proteinopathy, such as ALS, FTLD, AD. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

Relevant physiological or cellular responses (in vivo or in vitro) will be apparent to persons skilled in the art and may include maintaining the amount of stathmin-2 expression and includes preventing the downregulation of stathmin-2. “Maintaining” the amount is typically a statistically significant amount and may include stathmin-2 levels that have not decreased. Preferably the level of stathmin-2 has not decreased wherein the decrease is 1.1, 1.2, 2, 3, 4, 5, 6, 7, 8, 9, 10, 15, 20, 30, 40, 50 or less times (e.g., 500, 1000 times) (including all integers and decimal points in between and above 1, e.g., 1.5, 1.6, 1.7. 1.8) the stathmin-2 amount produced by no AON (the absence of an agent) or a control compound. Preferably the stathmin-2 expression has not reduced to a level to cause an adverse relevant physiological or cellular response, leading to a symptom of a disease or condition such as ALS, FTLD, AD described herein, as measured according to routine techniques in the diagnostic art. In one embodiment, after administration of an STMN2 AON, stathmin-2 expression increases. In a further embodiment, after administration of an STMN2 AON, stathmin-2 expression increases before preventing any further depletion or preventing depletion caused by the TDP43 AON.

### Modified AONs

In some embodiments, the AONs have the chemical composition of a naturally occurring nucleic acid molecule, i.e., the AONs do not include a modified or substituted base, sugar, or inter-subunit linkage.

In a preferred embodiment, the AONs of the present invention are non-naturally occurring nucleic acid molecules, or “oligonucleotide analogs”. For example, non-naturally occurring nucleic acids can include one or more non-natural base, sugar, and/or inter-subunit linkage, e.g., a base, sugar, and/or linkage that has been modified or substituted with respect to that found in a naturally occurring nucleic acid molecule. Exemplary modifications are described below. In some embodiments, non-naturally occurring nucleic acids include more than one type of modification, e.g., sugar and base modifications, sugar and linkage modifications, base and linkage modifications, or base, sugar, and linkage modifications. For example, in some embodiments, the AONs contain a non-natural (e.g., modified or substituted) base. In some embodiments, the AONs contain a non-natural (e.g., modified or substituted) sugar. In some embodiments, the AONs contain a non-natural (e.g., modified or substituted) inter-subunit linkage. In some embodiments, the AONs contain more than one type of modification or substitution, e.g., a non-natural base and/or a non-natural sugar, and/or a non-natural inter-subunit linkage.

Thus, included are non-naturally occurring AONs having (i) a modified backbone structure, e.g., a backbone other than the standard phosphodiester linkage found in naturally occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the invention will be aware of other forms of suitable backbones that may be useable in the objectives of the invention.

To avoid degradation of pre-RNA during duplex formation with the antisense oligomers, the AONs used in the method may be adapted to minimise or prevent cleavage by endogenous Rnase H. Antisense molecules that do not activate Rnase H can be made in accordance with known techniques (see, e.g., U.S. Pat. No. 5,149,797). Such antisense molecules, which may be deoxyribonucleotide or ribonucleotide sequences, simply contain any structural modification which sterically hinders or prevents binding of Rnase H to a duplex molecule containing the oligonucleotide as one member thereof, which structural modification does not substantially hinder or disrupt duplex formation. Because the portions of the oligonucleotide involved in duplex formation are substantially different from those portions involved in Rnase H binding thereto, numerous antisense molecules that do not activate Rnase H are available. This property is highly preferred, as the treatment of the RNA with the unmethylated oligomers, either intracellular or in crude extracts that contain Rnase H, leads to degradation of the pre-mRNA:AON duplexes. Any form of modified AONs that is capable of by-passing or not inducing such degradation may be used in the present method. The nuclease resistance may be achieved by modifying the AONs of the invention so that it comprises partially unsaturated aliphatic hydrocarbon chain and one or more polar or charged groups including carboxylic acid groups, ester groups, and alcohol groups.

An example of AONs which when duplexed with RNA are not cleaved by cellular Rnase H is 2′-O-methyl derivatives. Such 2′-O-methyl-oligoribonucleotides are stable in a cellular environment and in animal tissues, and their duplexes with RNA have higher Tm values than their ribo- or deoxyribo-counterparts. Alternatively, the nuclease resistant AONs of the invention may have at least one of the last 3′-terminus nucleotides fluoridated. Still alternatively, the nuclease resistant AONs of the invention have phosphorothioate bonds linking between at least two of the last 3-terminus nucleotide bases, preferably having phosphorothioate bonds linking between the last four 3′-terminal nucleotide bases.

Decreased RNA cleavage may also be achieved with alternative oligonucleotide chemistry (see, e.g., U.S. Pat. No. 5,149,797). For example, the AON may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′-O-Methyl-modified oligomer (2′-Ome); 2′-O-methoxy ethyl (2′-MOE); 2′-fluoro, 2′-fluroarabino (FANA); unlocked nucleic acid (UNA); hexitol nucleic acid (HNA); cyclohexenyl nucleic acid (CeNA); 2′-amino (2′-NH2); 2′-O-ethyleneamine or any combination of the foregoing as mixmers or as gapmers. The key benefit of PMOs is an increased safety profile. Their neutral charge makes them less susceptible to protein interactions with reduced platelet activation and immune activation. This also reduces degradation by nucleases. They have also been used safely in DMD patients for more than 5 years.

In an aspect, the modified AON of the invention can be conjugated to a peptide. Preferably, the AON is a PPMO, i.e., a PMO oligonucleotide chemically conjugated to a peptide moiety via amide, maleimide or click chemistry (preferably using copper-free click chemistry for example via cyclooctyne linkage) and includes suitable linkers, such as cleavable or pH-sensitive linkers. The peptide moiety may be linked via either the 3′ or the 5′ terminus. Most preferably, the peptide moiety is a peptide that is capable of improving the capacity of the AON to penetrate the cell and reach the nucleus. For example, the peptide moiety can be an arginine-rich peptide, cationic peptide and/or a peptide selected from a library of peptides derived from genomes of biodiverse microorganisms (Hoffman et al., Sci Rep, 8, 1, 12538). The peptides may or may not contain non-natural amino acids and/or chemically modified amino acids.

Cell penetrating peptides have been added to phosphorodiamidate morpholino oligomers to enhance cellular uptake and nuclear localization. Different cell penetrating peptides have been shown to influence efficiency of uptake and target tissue specificity, as shown in Jearawiriyapaisarn et al. (2008), Mol. Ther., 16(9), 1624-1629. The terms “cell penetrating peptide” and “CPP” are used interchangeably and refer to cationic cell penetrating peptides, also called transport peptides, carrier peptides, or peptide transduction domains. The peptides, as shown herein, have the capability of inducing cell penetration within 100% of cells of a given cell culture population and allow macromolecular translocation within multiple tissues in vivo upon systemic administration. The peptides are also capable of enhancing cellular uptake after localized delivery to a tissue or organ.

To further improve the delivery efficacy, the abovementioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct AONs to induce exon skipping. Antisense oligomer-induced alternative splicing of the human TARDBP gene transcripts can use oligoribonucleotides, PNAs, 2′-Ome or 2′-MOE modified bases on a phosphorothioate backbone. Although 2′-Ome AONs are used for oligo design, due to their efficient uptake in vitro when delivered as cationic lipoplexes, these compounds are susceptible to nuclease degradation and are not considered ideal for in vivo or clinical applications. When alternative chemistries are used to generate the AONs of the present invention, the uracil (U) of the sequences provided herein may be replaced by a thymine (T).

For example, such antisense molecules may be oligonucleotides wherein at least one, or all, of the inter-nucleotide bridging phosphate residues are modified phosphates, such as methyl phosphonates, methyl phosphorothioates, phosphoromorpholidates, phosphoropiperazidates and phosphor amidates. For example, every other one of the internucleotide bridging phosphate residues may be modified as described. In another non-limiting example, such antisense molecules are molecules wherein at least one, or all, of the nucleotides contain a 2′ lower alkyl moiety (e.g., Ci-C4, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of AONs useful in this invention include oligonucleotides containing modified backbones or non-natural intersubunit linkages.

Oligonucleotides having modified backbones include those that retain a phosphorus atom in the backbone and those that do not have a phosphorus atom in the backbone. Modified oligonucleotides that do not have a phosphorus atom in their inter-nucleoside backbone can also be considered to be oligonucleosides.

In other antisense molecules, both the sugar and the inter-nucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleo-bases are retained and are bound directly or indirectly to aza nitrogen atoms of the amide portion of the backbone.

Modified oligonucleotides may also contain one or more substituted sugar moieties. Oligonucleotides may also include nucleobase (often referred to in the art simply as “base”) modifications or substitutions. Oligonucleotides containing a modified or substituted base include oligonucleotides in which one or more purine or pyrimidine bases most commonly found in nucleic acids are replaced with less common or non-natural bases.

Purine bases comprise a pyrimidine ring fused to an imidazole ring; adenine and guanine are the two purine nucleobases most commonly found in nucleic acids. These may be substituted with other naturally occurring purines, including but not limited to N₆-methyladenine, N₂-methylguanine, hypoxanthine, and 7-methylguanine.

Pyrimidine bases comprise a six-membered pyrimidine ring; cytosine, uracil, and thymine are the pyrimidine bases most commonly found in nucleic acids. These may be substituted with other naturally occurring pyrimidines, including but not limited to 5-methylcytosine, 5-hydroxymethylcytosine, pseudouracil, and 4-thiouracil. In one embodiment, the oligonucleotides described herein contain thymine bases in place of uracil.

Other modified or substituted bases include, but are not limited to, 2,6-diaminopurine, orotic acid, agmatidine, lysidine, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N₂-cyclopentylguanine (cPent-G), N₂-cyclopentyl-2-aminopurine (cPent-AP), and N₂-propyl-2-aminopurine (Pr-AP), pseudouracil or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences). cPent-G, cPent-AP and Pr-AP were shown to reduce immunostimulatory effects when incorporated in siRNA (Peacock H. et al. J. Am. Chem. Soc. 2011, 133, 9200). Pseudouracil is a naturally occurring isomerized version of uracil, with a C-glycoside rather than the regular N-glycoside as in uridine. Pseudouridine-containing synthetic mRNA may have an improved safety profile compared to uridine-containing mPvNA (see WO 2009127230).

Certain modified or substituted nucleo-bases are particularly useful for increasing the binding affinity of the AONs of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently preferred base substitutions, even more particularly when combined with 2′-O-methoxyethyl sugar modifications.

In some embodiments, modified or substituted nucleo-bases are useful for facilitating purification of AONs. For example, in certain embodiments, AONs may contain three or more (e.g., 3, 4, 5, 6 or more) consecutive guanine bases. In certain AONs, a string of three or more consecutive guanine bases can result in aggregation of the oligonucleotides, complicating purification. In such AONs, one or more of the consecutive guanines can be substituted with inosine. The substitution of inosine for one or more guanines in a string of three or more consecutive guanine bases can reduce aggregation of the AON, thereby facilitating purification.

In one embodiment, another modification of the AONs involves chemically linking to the oligonucleotide one or more moieties or conjugates that enhance the activity, cellular distribution or cellular uptake of the oligonucleotide. Such moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-36lycerol-3-H-phosphonate, a polyamine or a polyethylene glycol chain, or adamantane acetic acid, a palmityl moiety, or an octadecylamine or hexylamino-carbonyl-oxycholesterol moiety.

It is not necessary for all positions in a given compound to be uniformly modified, and in fact more than one of the aforementioned modifications may be incorporated in a single compound or even at a single nucleoside within an oligonucleotide. The present invention also includes AONs that are chimeric compounds. “Chimeric” antisense compounds or “chimeras,” in the context of this invention, are antisense molecules, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the increased resistance to nuclease degradation, increased cellular uptake, and an additional region for increased binding affinity for the target nucleic acid.

The antisense molecules used in accordance with this invention may be conveniently and routinely made through the well-known technique of solid phase synthesis. Equipment for such synthesis is sold by several vendors including, for example, Applied Biosystems (Foster City, Calif.). One method for synthesising oligonucleotides on a modified solid support is described in U.S. Pat. No. 4,458,066.

In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

While the AONs described above are a preferred form of the AONs of the present invention, the present invention includes other oligomeric antisense molecules, including but not limited to oligomer mimetics such as are described below.

Another preferred chemistry is the phosphorodiamidate morpholino oligomer (PMO) oligomeric compounds, which are not degraded by any known nuclease or protease. These compounds are uncharged, do not activate RNase H activity when bound to an RNA strand and have been shown to exert sustained cleavage factor binding modulation after in vivo administration (Summerton and Weller, Antisense Nucleic Acid Drug Development, 1997; 7, 187-197). Preferably, the AONs of the invention are phosphorodiamidate morpholino oligomers.

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds of the invention. These include 5-substituted pyrimidines, 6-azapyrimidines, and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C., even more particularly when combined with 2′-O-methoxyethyl sugar modifications. In one embodiment, at least one pyrimidine base of the oligonucleotide comprises a 5-substituted pyrimidine base, wherein the pyrimidine base is selected from the group consisting of cytosine, thymine and uracil. In one embodiment, the 5-substituted pyrimidine base is 5-methylcytosine. In another embodiment, at least one purine base of the oligonucleotide comprises an N-2, N-6 substituted purine base. In one embodiment, the N-2, N-6 substituted purine base is 2,6-diaminopurine.

In one embodiment, the AON includes one or more 5-methylcytosine substitutions alone or in combination with another modification, such as 2′-O-methoxyethyl sugar modifications. In yet another embodiment, the AON includes one or more 2,6-diaminopurine substitutions alone or in combination with another modification.

In some embodiments, the AON is chemically linked to one or more moieties, such as a polyethylene glycol moiety, or conjugates, such as an arginine-rich cell penetrating peptide that enhance the activity, cellular distribution, or cellular uptake of the AON. In one exemplary embodiment, the arginine-rich polypeptide is covalently coupled at its N-terminal or C-terminal residue to the 3′ or 5′ end of the antisense compound. Also, in an exemplary embodiment, the antisense compound is composed of morpholino subunits and phosphorus-containing inter-subunit linkages joining a morpholino nitrogen of one subunit to a 5′ exocyclic carbon of an adjacent subunit.

In another aspect, the invention provides expression vectors that incorporate the AONs described above, e.g., the AONs of SEQ ID NOs: 1-66. In some embodiments, the expression vector is a modified retrovirus or non-retroviral vector, such as an adeno-associated viral vector.

Assays for measuring activity of AONs

The activity of AONs and variants thereof can be assayed according to routine techniques in the art. For example, isoform forms and expression levels of surveyed RNAs and proteins may be assessed by any of a wide variety of well-known methods for detecting isoforms and/or expression of a transcribed nucleic acid or protein. Non-limiting examples of such methods include RT-PCR of isoforms of RNA followed by size separation of PCR products, nucleic acid hybridization methods e.g., Northern blots and/or use of nucleic acid arrays; fluorescent in situ hybridization to detect RNA transcripts inside cells; nucleic acid amplification methods; immunological methods for detection of proteins; protein purification methods; and protein function or activity assays.

RNA expression levels can be assessed by preparing RNA/cDNA (i.e., a transcribed polynucleotide) from a cell, tissue or organism, and by hybridizing the RNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

Methods of manufacturing AONs

The AONs used in accordance with this invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs of the invention are synthesised in vitro and do not include antisense compositions of biological origin, or genetic vector constructs designed to direct the in vivo synthesis of antisense oligomers. The molecules of the invention may also be mixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption.

### Vectors

Also included are vector delivery systems that are capable of expressing the oligomeric, TARDBP-targeting sequences of the present invention, such as vectors that express a polynucleotide sequence comprising any one or more of SEQ ID NO: 1 to SEQ ID NO: 25, SEQ ID NO: 38 to 58, as described herein.

Also included are vector delivery systems that are capable of expressing the oligomeric, STMN2-targeting sequences of the present invention, such as vectors that express a polynucleotide sequence comprising any one or more of SEQ ID NO: 29 to SEQ ID NO: 37, SEQ ID NO: 62 to 66, as described herein.

By “vector” or “nucleic acid construct” is meant a polynucleotide molecule, preferably a DNA molecule derived, for example, from a plasmid, bacteriophage, yeast or virus, into which a polynucleotide can be inserted or cloned. A vector preferably contains one or more unique restriction sites and can be capable of autonomous replication in a defined host cell including a target cell or tissue or a progenitor cell or tissue thereof or able to be integrated with the genome of the defined host such that the cloned sequence is reproducible.

Accordingly, the vector can be an autonomously replicating vector, i.e., a vector that exists as an extra-chromosomal entity, the replication of which is independent of chromosomal replication, e.g., a linear or closed circular plasmid, an extra-chromosomal element, a mini-chromosome, or an artificial chromosome. The vector can contain any means for assuring self-replication. Alternatively, the vector can be one which, when introduced into the host cell, is integrated into the genome and replicated together with the chromosome(s) into which it has been integrated.

### C. Method of Treatment

The AONs of the present invention also can be used as a prophylactic or therapeutic, which may be utilised for the purpose of treatment of a disease. Accordingly, in one embodiment the present invention provides AONs that bind to a selected target in the TARDBP pre-mRNA to reduce expression of TARDBP as described herein, in a therapeutically effective amount, admixed with a pharmaceutically acceptable carrier, diluent, or excipient.

TDP43 is produced in the cytoplasm and imported into the nucleus. Aggregations of TDP43 in the cytoplasm can deplete TDP43 availability for nuclear trafficking, which can result in autoregulation causing the upregulation of TARDBP mRNA, resulting in increased TDP43. Without being bound by theory, decreasing the expression of TARDBP mRNA through the administration of the AONs of this invention, may result in a reduction in TDP43 in aggregates and thus the improvement of TDP43 nuclear trafficking as more TDP43 is expected to be available for trafficking rather than accumulated in aggregates.

An “effective amount” or “therapeutically effective amount” refers to an amount of therapeutic compound, such as an antisense oligomer, administered to a mammalian subject, either as a single dose or as part of a series of doses, which is effective to produce a desired therapeutic effect.

The invention therefore provides a pharmaceutical, prophylactic, or therapeutic composition to treat, prevent or ameliorate the effects of a disease associated with TDP43 proteinopathy, the composition comprising:
a) one or more AONs as described herein, and
b) one or more pharmaceutically acceptable carriers and/or diluents.

Preferably, the disease associated with TDP43 proteinopathy is ALS, FTLD or AD.

There is also provided a method for treating, preventing or ameliorating the effects of a disease associated with TDP43 proteinopathy, the method comprising the step of: administering to the subject an effective amount of one or more AONs or pharmaceutical composition comprising one or more AONs as described herein.

The invention provides a method for treating, preventing or ameliorating the effects of a disease associated with TDP43 proteinopathy, the method comprising the step of: administering to the subject an effective amount of one or more AONs or pharmaceutical composition comprising one or more AONs as described herein.

There is also provided a method for treating, preventing or ameliorating the effects of ALS, the method comprising the step of: administering to the subject an effective amount of one or more AONs or pharmaceutical composition comprising one or more AONs as described herein.

The methods of the invention can be administered in combination with additional treatments for treating, preventing, or slowing the progress of diseases associated with TDP43 proteinopathy and their symptoms. Additional treatments can include AONs directed to other targets associated with diseases associated with TDP43 proteinopathy. For example, the additional treatments can include AONs directed to SOD1.

In a further aspect, genetic or other biomarkers can be used to identify patients most likely to respond well to TDP43 suppression via the AONs of the invention. Genetic structural variations associated with ALS disease risk have been identified within ALS genes and surrounding gene regions. These variations can be used as genetic biomarkers to identify patients likely to respond to the methods of this invention. Non-genetic biomarkers can also be used to identify patients likely to respond to the methods of this invention. Truncated STMN2 has also been found to be a marker of TDP43 pathology in FTD (Prudencio et. al. J Clin Invest. 2020 130(11): 6080-6092). Preferably, the biomarker is a form of truncated STMN2.

The invention provides a method for treating, preventing or ameliorating the effects of ALS, FTLD or AD, in subjects identified by a biomarker, the method comprising the step of:
a) testing a subject for the presence of a biomarker associated with ALS patients likely to respond to TDP43 suppression; and
b) if the subject is found to express the biomarker, administering to the subject an effective amount of one or more AONs or pharmaceutical composition comprising one or more AONs as described herein.

There is also provided herein the use of purified and isolated AONs as described herein, to treat, prevent or ameliorate the effects of a disease associated with TDP43 proteinopathy.

There is also provided herein the use of purified and isolated AONs as described herein, to treat, prevent or ameliorate the effects of ALS, FTLD or AD.

Preferably, the AON used in the present invention is chosen from the list of AONs provided in Tables 1 or 2 or more preferably is selected from SEQ ID NO: 12, 16, 24, or 25.

The invention also provides a method of treatment that comprises the combination of: (1) AONs designed to reduce TDP43 expression (SEQ ID NO: 1 to SEQ ID NO: 25, SEQ ID NO: 38 to 58); with (2) AONs designed to prevent the subsequent downregulation of stathmin-2 (SEQ ID NO: 29 to SEQ ID NO: 37, SEQ ID NO: 62 to 66), to reduce the impact of cytoplasmic TDP43 overexpression whilst protecting against the neuronal vulnerability caused by stathmin-2 depletion. In one embodiment, the invention seeks to provide a means for ameliorating TDP43 proteinopathy in subjects suffering from diseases associated with TDP43 proteinopathy whilst maintaining normal physiological levels of or reducing downregulation of stathmin-2.

Preferably, the AON designed to prevent the subsequent downregulation of stathmin-2 is selected from the list of SEQ ID NO: 29 to SEQ ID NO: 37, SEQ ID NO: 62 to 66, or more preferably is selected from SEQ ID NO: 33, 35, 36 and 37.

The composition may comprise about 1 nM to 1000 μM of each of the desired antisense oligomer(s) of the invention. Preferably, the composition may comprise about 1 μM to 500 μM, 10 μM to 500 μM, 50 μM to 750 μM, 10 μM to 500 μM, 1 μM to 100 μM, 1 μM to 50 μM, preferably between 25 μM and 100 μM of each of the antisense oligomer(s) of the invention. The composition may also preferably comprise about 1 nM to 500 nM, 10 nM to 500 nM, 50 nM to 750 nM, 10 nM to 500 nM, 1 nM to 100 nM, 1 nM to 50 nM, most preferably between 50 nM and 100 nM of each of the antisense oligomer(s) of the invention.

The composition may comprise about 1 nM, 2 nM, 3 nM, 4 nM, 5 nM, 6 nM, 7 nM, 8 nM, 9 nM, 10 nM, 20 nM, 50 nM, 75 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM or 1000 nM of each of the desired antisense oligomer(s) of the invention.

The present invention further provides one or more AONs adapted to aid in the prophylactic or therapeutic treatment, prevention or amelioration of symptoms of a disease or pathology associated with TDP43 proteinopathy in a form suitable for delivery to a subject.

The phrase “pharmaceutically acceptable” refers to molecular entities and compositions that are physiologically tolerable and do not typically produce an allergic or similarly untoward reaction, such as gastric upset and the like, when administered to a subject. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

The pharmaceutical composition comprising the one or more AONs can be administered to the subject in a range of treatment regimens. For example, the pharmaceutical composition can be administered hourly, three times daily, twice daily, once daily, once every two days, once every three days, once weekly, once every two weeks, once monthly, once every two months, once every six months, and once yearly. The appropriate regimen can be determined by the person skilled in the art based on the nature of the condition to be treated.

### D. Manufacture of a Medicament

In one embodiment, the present invention provides the use of AONs that bind to a selected target in the TARDBP RNA for the manufacture of a medicament to treat, prevent or ameliorate the effects of a disease associated with TDP43 proteinopathy. There is therefore provided the use of one or more AONs described herein for the manufacture of a medicament to treat, prevent or ameliorate the effects of a disease associated with TDP43 proteinopathy. Preferably the disease is ALS, FTLD, FTD or AD.

The invention provides the use of purified and isolated antisense oligonucleotides according as described herein, for the manufacture of a medicament to treat, prevent or ameliorate the effects of a disease associated with a disease associated with TDP43 proteinopathy.

The invention also provides the use of purified and isolated antisense oligonucleotides according as described herein, for the manufacture of a medicament to treat, prevent or ameliorate the effects of ALS, FTLD or AD.

There is also provided the use of one or more AONs described herein for the manufacture of a medicament to treat, prevent or ameliorate the effects of a disease associated with TDP43 proteinopathy in subjects expressing a biomarker associated with patients likely to respond to TDP43 suppression.

Preferably, the AON used for the manufacture of a medicament is chosen from the list of AONs provided in Tables 1 or 2 or more preferably is selected from SEQ ID NO: 12, 16, 24, or 25.

In a further embodiment, the present invention also provides the use of AONs that bind to a selected target in the STMN2 RNA for the manufacture of a medicament to maintain normal physiological levels of or reduce downregulation of stathmin-2. Preferably, the AON used is chosen from the list of AONs provided in Tables 1 or 2 or more preferably is selected from SEQ ID NO: 29 to SEQ ID NO: 37, SEQ ID NO: 62 to 66. Most preferably, the AON is selected from SEQ ID NO: 33, 35, 36 and 37.

### E. Pharmaceutical Compositions

In a form of the invention there are provided pharmaceutical compositions comprising therapeutically effective amounts of one or more AONs of the invention together with pharmaceutically acceptable diluents, preservatives, solubilizers, emulsifiers, adjuvants, and/or carriers. Such compositions include diluents of various buffer content (e.g., Tris-HCl, acetate, phosphate), pH and ionic strength and additives such as detergents and solubilizing agents (e.g., Tween 80, Polysorbate 80), antioxidants (e.g., ascorbic acid, sodium metabisulfite), preservatives (e.g., Thimersol, benzyl alcohol) and bulking substances (e.g., lactose, mannitol). The material may be incorporated into particulate preparations of polymeric compounds such as polylactic acid, polyglycolic acid, etc. or into liposomes. Hyalluronic acid may also be used. Such compositions may influence the physical state, stability, rate of in vivo release, and rate of in vivo clearance of the present proteins and derivatives. See, for example, Martin, Remington's Pharmaceutical Sciences, 18th Ed. (1990, Mack Publishing Co., Easton, PA 18042) pages 1435-1712 that are herein incorporated by reference. The compositions may be prepared in liquid form, or may be in dried powder, such as a lyophilised form.

It will be appreciated that pharmaceutical compositions provided according to the present invention may be administered by any means known in the art. Preferably, the pharmaceutical compositions for administration are administered by injection, orally, topically or by the pulmonary or nasal route. The AONs are more preferably delivered by intravenous, intrathecal, intra-arterial, intraperitoneal, intramuscular or subcutaneous routes of administration. The appropriate route may be determined by one of skill in the art, as appropriate to the condition of the subject under treatment. Vascular or extravascular circulation, the blood or lymph system, and the cerebrospinal fluid are some non-limiting sites where the AON may be introduced. Direct CNS delivery may be employed, for instance, intracerebro-ventricular or intrathecal administration may be used as routes of administration.

Formulations for topical administration include those in which the oligomers of the disclosure are in admixture with a topical delivery agent such as lipids, liposomes, fatty acids, fatty acid esters, steroids, chelating agents and surfactants. Lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). For topical or other administration, oligomers of the disclosure may be encapsulated within liposomes or may form complexes thereto, in particular to cationic liposomes. Alternatively, oligomers may be complexed to lipids, in particular to cationic lipids. Fatty acids and esters, pharmaceutically acceptable salts thereof, and their uses are further described in U.S. Pat. No. 6,287,860 and/or U.S. patent application Ser. No. 09/315,298 filed on May 20, 1999.

In certain embodiments, the AONs of the disclosure can be delivered by transdermal methods (e.g., via incorporation of the AONs into, e.g., emulsions, with such AONs optionally packaged into liposomes). Such transdermal and emulsion/liposome-mediated methods of delivery are described for delivery of AONs in the art, e.g., in U.S. Pat. No. 6,965,025.

The AONs described herein may also be delivered via an implantable device. Design of such a device is an art-recognized process, with, e.g., synthetic implant design described in, e.g., U.S. Pat. No. 6,969,400.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Oral formulations are those in which oligomers of the disclosure are administered in conjunction with one or more penetration enhancers surfactants and chelators. Surfactants include fatty acids and/or esters or salts thereof, bile acids and/or salts thereof. Bile acids/salts and fatty acids and their uses are further described in U.S. Pat. No. 6,287,860. In some embodiments, the present disclosure provides combinations of penetration enhancers, for example, fatty acids/salts in combination with bile acids/salts. An exemplary combination is the sodium salt of lauric acid, capric acid and UDCA. Further penetration enhancers include polyoxyethylene-9-lauryl ether, polyoxyethylene-20-cetyl ether. Oligomers of the disclosure may be delivered orally, in granular form including sprayed dried particles, or complexed to form micro or nanoparticles. Oligomer complexing agents and their uses are further described in U.S. Pat. No. 6,287,860. Oral formulations for oligomers and their preparation are described in detail in U.S. Pat. No. 6,887,906, U.S. Ser. No. 09/315,298 filed May 20, 1999 and/or US20030027780.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98(1) 42-47, and in Gebski et al. (2003) Human Molecular Genetics, 12(15): 1801-1811. A method for introducing a nucleic acid molecule into a cell by way of an expression vector either as naked DNA or complexed to lipid carriers, is described in U.S. Pat. No. 6,806,084.

In certain embodiments, the AONs of the invention and therapeutic compositions comprising the same can be delivered by transdermal methods (e.g., via incorporation of the AONs into, e.g., emulsions, with such AONs optionally packaged into liposomes). Such transdermal and emulsion/liposome-mediated methods of delivery are described for delivery of AONs in the art, e.g., in U.S. Pat. No. 6,965,025.

It may be desirable to deliver the AON in a colloidal dispersion system. Colloidal dispersion systems include macromolecule complexes, nanocapsules, microspheres, beads, and lipid-based systems including oil-in-water emulsions, micelles, mixed micelles, and liposomes or liposome formulations. These colloidal dispersion systems can be used in the manufacture of therapeutic pharmaceutical compositions.

Liposomes are artificial membrane vesicles, which are useful as delivery vehicles in vitro and in vivo. These formulations may have net cationic, anionic, or neutral charge characteristics and have useful characteristics for in vitro, in vivo and ex vivo delivery methods. It has been shown that large unilamellar vesicles can encapsulate a substantial percentage of an aqueous buffer containing large macromolecules. RNA and DNA can be encapsulated within the aqueous interior and be delivered to cells in a biologically active form (Fraley, et al., 1981, Trends Biochem. Sci., 6, 77).

In order for a liposome to be an efficient gene transfer vehicle, the following characteristics should be present: (1) encapsulation of the AON of interest at high efficiency while not compromising their biological activity; (2) preferential and substantial binding to a target cell in comparison to non-target cells; (3) delivery of the aqueous contents of the vesicle to the target cell cytoplasm at high efficiency; and (4) accurate and effective expression of genetic information (Mannino, et aL, 1988 Biotechniques, 6, 682). The composition of the liposome is usually a combination of phospholipids, particularly high phase-transition-temperature phospholipids, usually in combination with steroids, especially cholesterol. Other phospholipids or other lipids may also be used. The physical characteristics of liposomes depend on pH, ionic strength, and the presence of divalent cations. Cationic liposomes are positively charged liposomes which are believed to interact with negatively charged DNA molecules to form a stable complex. Liposomes that are pH-sensitive or negatively charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

AONs can be introduced into cells using art-recognized techniques (e.g., transfection, electroporation, fusion, liposomes, colloidal polymeric particles and viral and non-viral vectors as well as other means known in the art). The method of delivery selected will depend at least on the cells to be treated and the location of the cells and will be apparent to the skilled artisan. For instance, localization can be achieved by liposomes with specific markers on the surface to direct the liposome, direct injection into tissue containing target cells, specific receptor-mediated uptake, or the like.

As known in the art, AONs may be delivered using, for example, methods involving liposome-mediated uptake, lipid conjugates, polylysine-mediated uptake, nanoparticle-mediated uptake, and receptor-mediated endocytosis, as well as additional non-endocytic modes of delivery, such as microinjection, permeabilization (e.g., streptolysin-O permeabilization, anionic peptide permeabilization), electroporation, and various non-invasive non-endocytic methods of delivery that are known in the art (refer to Dokka and Rojanasakul, Advanced Drug Delivery Reviews 44, 35-49, incorporated by reference in its entirety).

The AON may also be combined with other pharmaceutically acceptable carriers or diluents to produce a pharmaceutical composition. Suitable carriers and diluents include isotonic saline solutions, for example phosphate-buffered saline. The composition may be formulated for parenteral, intramuscular, intravenous, subcutaneous, intraocular, oral, or transdermal administration.

The routes of administration described are intended only as a guide since a skilled practitioner will be able to readily determine the optimum route of administration and any dosage for any particular animal and condition.

Multiple approaches for introducing functional new genetic material into cells, both in vitro and in vivo have been attempted (Friedmann (1989) Science, 244, 1275-1280). These approaches include integration of the gene to be expressed into modified retroviruses (Friedmann (1989) supra; Rosenberg (1991) Cancer Research 51(18), suppl.: 5074S-5079S); integration into non-retrovirus vectors (Rosenfeld, et al. (1992) Cell, 68, 143-155; Rosenfeld, et al. (1991) Science, 252, 431-434); or delivery of a transgene linked to a heterologous promoter-enhancer element via liposomes (Friedmann (1989), supra; Brigham, et al. (1989) Am. J. Med. Sci., 298, 278-281; Nabel, et al. (1990) Science, 249, 1285-1288; Hazinski, et al. (1991) Am. J. Resp. Cell Molec. Biol., 4:206-209; and Wang and Huang (1987) Proc. Natl. Acad. Sci. (USA), 84, 7851-7855); coupled to ligand-specific, cation-based transport systems (Wu and Wu (1988) J. Biol. Chem., 263, 14621-14624) or the use of naked DNA, expression vectors (Nabel et al. (1990), supra); Wolff et al. (1990) Science, 247, 1465-1468). Direct injection of transgenes into tissue produces only localized expression (Rosenfeld (1992) supra); Rosenfeld et al. (1991) supra; Brigham et al. (1989) supra; Nabel (1990) supra; and Hazinski et al. (1991) supra). The Brigham et al. group ((1989) Am. J. Med. Sci. 298, 278-281 and Clinical Research (1991) 39 (abstract)) have reported in vivo transfection only of lungs of mice following either intravenous or intratracheal administration of a DNA liposome complex. An example of a review article of human gene therapy procedures is: Anderson, (1992) Science 256, 808-813; Barteau et al. (2008), Curr Gene Ther., 8(5), 313-23; Mueller et al. (2008). Clin Rev Allergy Immunol., 35(3), 164-78; Li et al. (2006) Gene Ther., 13(18), 1313-9; Simoes et al. (2005) Expert Opin Drug Deliv., 2(2), 237-54.

The AONs of the invention encompass any pharmaceutically acceptable salts, esters, or salts of such esters, or any other compound which, upon administration to an animal including a human, is capable of providing (directly or indirectly) the biologically active metabolite or residue thereof. Accordingly, as an example, the disclosure is also drawn to prodrugs and pharmaceutically acceptable salts of the compounds of the invention, pharmaceutically acceptable salts of such pro-drugs, and other bioequivalents.

The term “pharmaceutically acceptable salts” refers to physiologically and pharmaceutically acceptable salts of the compounds of the invention: i.e., salts that retain the desired biological activity of the parent compound and do not impart undesired toxicological effects thereto. For oligomers, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. The pharmaceutical compositions of the present invention may be administered in a number of ways depending upon whether local or systemic treatment is desired and upon the area to be treated. Administration may be topical (including ophthalmic and mucous membranes, as well as rectal delivery), pulmonary, e.g., by inhalation or insufflation of powders or aerosols (including by nebulizer, intratracheal, intranasal, epidermal and transdermal), oral or parenteral. Parenteral administration includes intravenous, intra-arterial, subcutaneous, intraperitoneal or intramuscular injection or infusion; or intracranial, e.g., intrathecal or intraventricular, administration. Oligomers with at least one 2′-O-methoxyethyl modification are believed to be particularly useful for oral administration. Preferably, the AON is delivered via the subcutaneous or intravenous route.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipients(s). In general, the formulations are prepared by uniformly and intimately bringing into association the active ingredients with liquid carriers or finely divided solid carriers or both, and then, if necessary, shaping the product.

The following Examples are to be construed as merely illustrative and not limitative of the remainder of the disclosure in any way whatsoever. These Examples are included solely for the purposes of exemplifying the present invention. They should not be understood as a restriction on the broad summary, disclosure or description of the invention as set out above. Without further elaboration, it is believed that one skilled in the art can, using the preceding description, utilize the present invention to its fullest extent. In the foregoing and in the following examples, all temperatures are set forth uncorrected in degrees Celsius; and, unless otherwise indicated, all parts and percentages are by weight.

### EXAMPLES

### Examples

### Example 1—Design of Original AONs

Exon 2 and exon 3 targeted splice switching AONs were designed to bind to exonic splice sites and splicing enhancer sites within the exons as predicted by online splice prediction tools. Skipping of either of these exons would lead to a shift in the reading frame of the transcript leading to a premature termination codon (TAA) in the following exon. Transcripts with premature termination codons are known to be decayed via nonsense mediated decay, an RNA surveillance mechanism that operates in all eukaryotic cells.

The sequences, gene co-ordinates, and SEQ IDs for the AONs are listed in Tables 1 and 2. AONs 1 to 66 (as provided in the Figures) correspond to SEQ ID Nos 1 to 66.

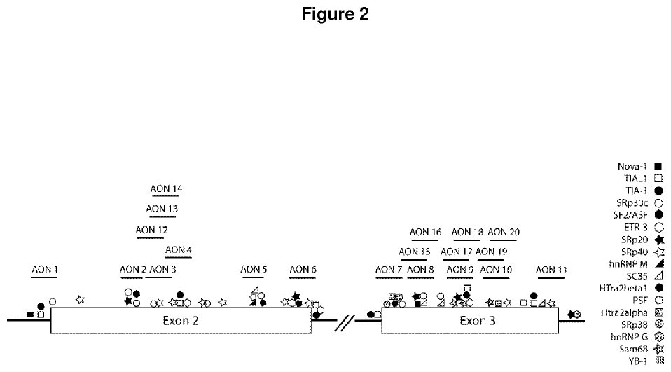

The AONs had 2′-O-Methyl sugar modifications and a phosphorothioate (PS) backbone chemistry. AON nomenclature was based on that described by Mann et al. (The Journal of Gene Medicine, 2002. 4(6): p. 644-654) whereby the species, gene, exon number, acceptor or donor targeting and annealing coordinates are described, where “−” indicates intronic position and “+” specifies exonic location from the splice site, as described herein. FIG. 2 presents a schematic diagram of the binding location of each of the AONs on TARDBP.

AONs with 2′-O-Methyl modifications and a PS backbone were ordered from TriLink Biotechnologies, Inc (San Diego, CA, USA) or ChemGenes Corporation (Wilmington, MA, USA). AONs with a phosphorodiamidate backbone (PMOs) were ordered from Genetools LLC (Philomath, OR, USA).

### Example 2—AON Screening Using Transcript Analysis

RT-PCR analysis of the TARDBP transcript was conducted following transfection of 2′-O-Methyl AONs. The level of TARDBP knockdown or exon skipping following AON transfection was compared to that of control treated and untreated samples.

Control sequences include an AON targeted to an unrelated gene, SMN, ctrl AON 1 (SEQ ID NO: 26, CACCUUCCUUCUUUUUGAUU) and negative control oligos purchased from GeneTools: ctrl AON 2 (SEQ ID NO: 27; GGAUGUCCUGAGUCUAGACCCUCCG) and ctrl AON 3 (SEQ ID NO: 28, CCTCTTACCTCAGTTACAATTTATA).

### Materials and Methods

### Transfection of Fibroblasts

Normal human dermal fibroblasts were propagated according to established techniques with 15,000 cells seeded into 24 well plates in 10% FBS DMEM and incubated at 37° C. for 24 hours prior to transfection. All 2′-O-Methyl PS-AOs were transfected using Lipofectamine 3000 (3 μl per ml of transfection volume) (Life Technologies, Melbourne, Australia), according to manufacturer's protocols, and AO transfected cells incubated for 24 hours.

### Transcript Analysis

RNA was extracted using the MagMAX-96 Total RNA Isolation Kit, including a DNase treatment (Life Technologies), according to the manufacturer's instructions. RT-PCRs were performed using the One-step Superscript Ill RT-PCR kit with Platinum Taq polymerase (Life Technologies) according to manufacturer's instructions. Products were amplified across TARDBP exons 1 to 6 (Fwd: CATTTTGTGGGAGCGAAGCG (SEQ ID NO: 67), Rev: ACGCACCAAAGTTCATCCCA (SEQ ID NO: 68)), with the temperature profile, 55° C. for 30 min, 94° C. for 2 min, followed by 24 cycles of 94° C. for 40 sec, 55° C. for 30 sec and 68° C. for 1 min 30 sec. Where applicable, results were normalised to transcript levels of an unrelated housekeeping control gene (TBP) amplified across exons 2 to 3 using the following primers (Fwd: AGCGCAAGGGTTTCTGGTTT (SEQ ID NO: 69), Rev: GGAGTCATGGGGGAGGGATA (SEQ ID NO: 70)).

PCR products were fractionated on 2% agarose gels in Tris-Acetate-EDTA buffer and the images captured on gel documentation system (Vilber Lourmat, Eberhardzell, Germany). Densitometric analysis was carried out using Image J. Exon skipping was quantitated by band weight to estimate ratios of full length TARDBP and exon skipped products. Product identity was confirmed by band purification and DNA sequencing as necessary. The efficiency of exon skipping was determined by calculating the percentage of the transcripts with exon(s) skipped compared to the total product generated by RT-PCR. The percentage of full-length transcript knockdown was determined by normalisation to a housekeeping gene and comparison of full-length transcript compared to control treated or untreated samples.

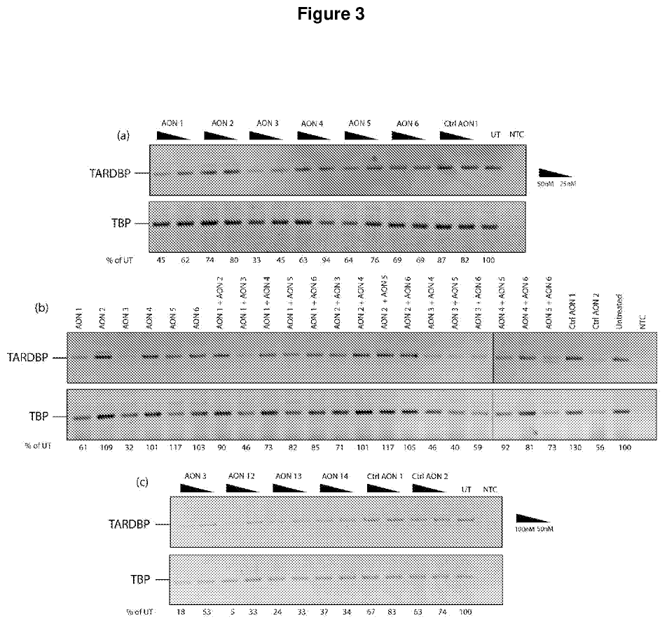

Exon 2 targeted AOs (AONs 1 to 6, SEQ ID NO ID 1 to 6) were first tested at 25 and 50 nM. No evidence of Exon 2 skipping was seen however a knockdown/reduction in the full-length transcript was detected in each of AONs 1 to 6 (SEQ ID NO: 1 to 6), with AON 3 (SEQ ID NO: 3) producing the greatest knockdown (FIG. 3a).

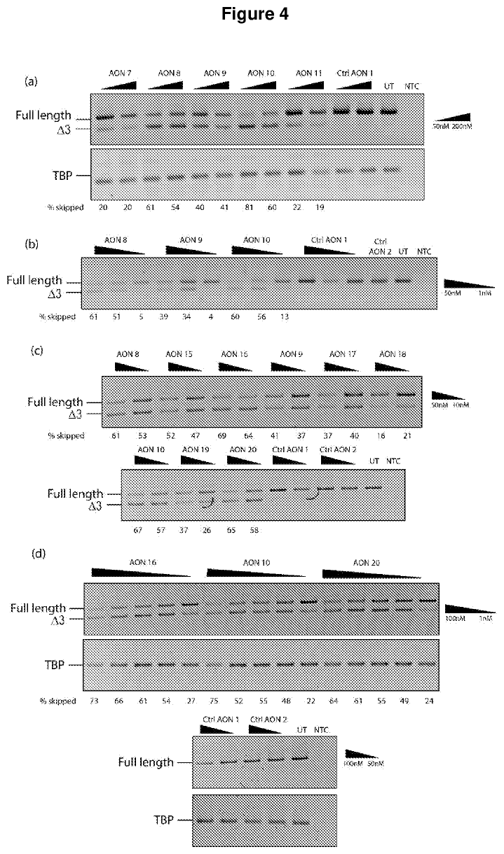

Exon 3 targeted AONs (AONs 7 to 11, SEQ ID NO ID 7 to 11) were first tested at 200 and 50 nM. All sequences tested were able to induce exon 3 skipping (FIG. 4a). AON 10 (SEQ ID NO: 10) was most effective with 81% or transcripts displaying exon 3 skipping when treated at 50 nM. The sequences that produced the greatest exon skipping (AONs 8, 9, and 10, SEQ ID NO: 8, 9 and 10) were then tested at a range of concentrations (50, 10 and 1 nM) with exon skipping seen down to 1 nM (FIG. 4b).

### Example 3—Optimisation of AONs

Following initial AON screening, AONs targeting TARDBP exon 2 were tested in cocktails (transfection of 2 AONs together at 50 nM of each) or as single AONs with no combination showing greater knockdown than AON 3 (SEQ ID NO 3) alone (FIG. 3b).

Following screening, AONs targeting TARDBP exons 2 and 3 were optimised by micro-walking (shifting the AON sequences up or down stream by several bases to identify the optimal target site, while still maintaining oligomer length) and the level of exon skipping following transfection was compared to the original AON sequence, control AONs and untreated samples.

Exon 2 targeted AON 3 (SEQ ID NO: 3) was micro-walked 5 bases upstream and 5 and 10 bases downstream to produce AONs 12, 13 and 14 (SEQ IDs 12, 13 and 14), and were tested at 50 and 100 nM concentrations with AON 12 (SEQ ID NO 12) producing greater knockdown of the TARDBP transcript that AON 3 (SEQ ID NO: 3) (FIG. 3c).

Exon 3 targeted AONs (AONs 8, 9 and 10, SEQ ID Nos 8, 9 and 10) were micro-walked 5 bases up and downstream (AONs 15 to 20, SEQ ID Nos 15 to 20) and were tested in several experiments at a range of concentrations to determine the lead molecule with AON 16 (SEQ ID NO 16) showing the greatest efficiency (FIGS. 4c and 4d).

### Example 4—Mechanism of Full-Length Transcript Knockdown for Exon 2 Targeted AONs

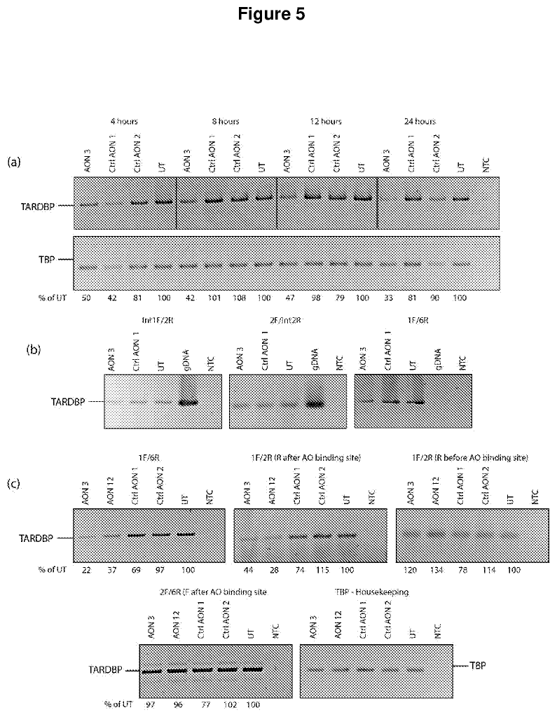

As no skipping of exon 2 was evident, the mechanism of action of the exon 2 targeted AONs were further explored. The result seen (transcript knockdown but no skipping) could have resulted from various causes. In some cases, exon skipping occurs, but the resulting transcripts are degraded so promptly or efficiently that it is difficult to detect when looking at one snapshot in time. In order to check if exon skipping was evident at earlier time points, a time course was undertaken with cells transfected at an AON concentration of 50 nM and cells collected at 4, 8, 12 and 24 hours after transfection. No exon skipping was seen at earlier time points. Knockdown could be seen from 4 hours after transfection (FIG. 5a).

Another possibility is that the AONs were causing an intron to be retained in the transcript. This could look like knockdown on a gel if the extension time in the PCR was not long enough to allow the transcripts containing the intron to be amplified. Primers were designed to look for intron retention however levels of the retained introns were uniform across treated, untreated and control treated cell extracts (FIG. 5b).

To further interrogate the mechanism of knockdown multiple strategically placed primer sets were utilised and transcript levels analysed. Knockdown is seen when the binding site is between the forward and reverse primer but not when both primers are before or after the binding site indicating that cleavage may be taking place close to the AON binding site (FIG. 5c).

### Example 5—PA1 Targeted AON Design and Screening

AONs were designed that would sterically inhibit the use of PA1 by blocking the polyadenylation signal. This could lead to a reduction of the translated transcript (1) and an increase in the untranslated transcripts (II, Ill, IV).

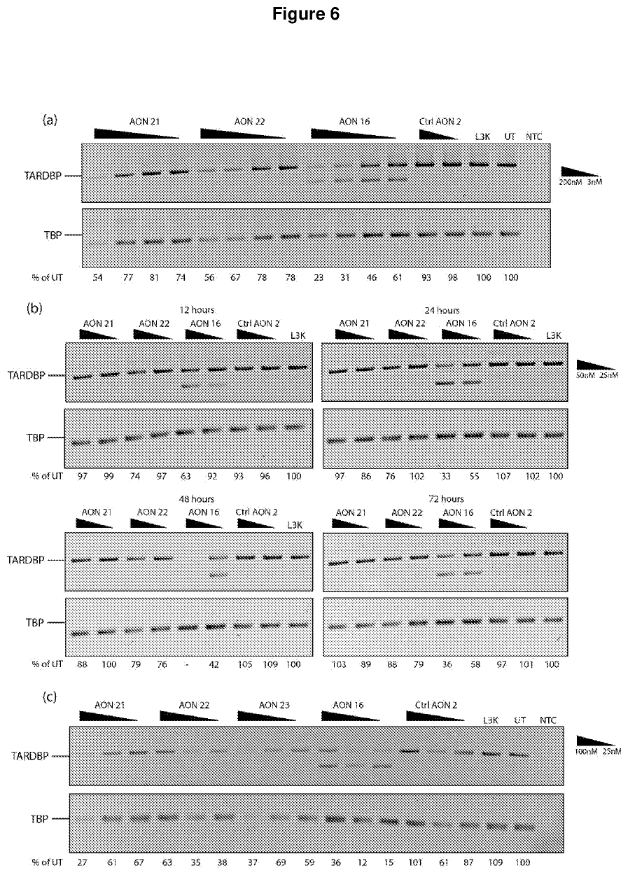

Two AONs of 20 and 25 bases length were initially synthesised using the 2′-O-Methyl PS chemistry to target PA1 of TARDBP (AON 21, 22, SEQ ID NO: 21 and 22). They were tested alongside lead exon skipping AON (SEQ ID NO 16) at a range of concentrations (200, 50, 12.5 and 3 nM) in human fibroblast cells as described in methods above. Some knockdown of the transcript was seen, however the level of knockdown was lower than with AON 16 (SEQ ID NO: 16) (FIG. 6a).

A time course was undertaken with cells transfected at 50 and 25 nM concentrations and RNA collected at 12, 24, 48 and 72 hours after transfection to see if knockdown was improved at an earlier or later timepoint. Though knockdown was observed, it was lower across all timepoints and was lower for the PA1 targeted AONs than the lead exon 3 skipping AON (FIG. 6b). An additional AON 30 bases in length was designed (AON 23, SEQ ID NO: 23) and tested in a final experiment at 100, 50 and 25 nM). Whilst a greater level of knockdown was seen for all 3 PA1 targeted AOs in this experiment the additional length did not lead to an increase in knockdown and some knockdown of the housekeeping gene was seen at higher concentrations (FIG. 6c).

### Example 6—Transcript and Protein Analysis of SEQ ID NO: 12 and 16 as PMOs

Exon 2 targeted AON 12 (SEQ ID NO: 12) and exon 3 targeted AON 16 (SEQ ID NO: 16) were synthesised and evaluated as PMOs (SEQ ID NO: 24 and SEQ ID NO: 25). The PMOs were delivered by nucleofection into normal fibroblasts, and then evaluated using RT-PCR as described above.

PMO delivery by nucleofection was performed using a Nucleofection X unit with the Nucleofection P2 kit, using the CA-137 program (Lonza, Melbourne, Australia). PMOs were initially transfected at 150 μM within the cuvette, supplemented with 5% FBS DMEM and incubated for 1, 3 and 5 days (RNA analysis) and 3 and 5 days (protein analysis).

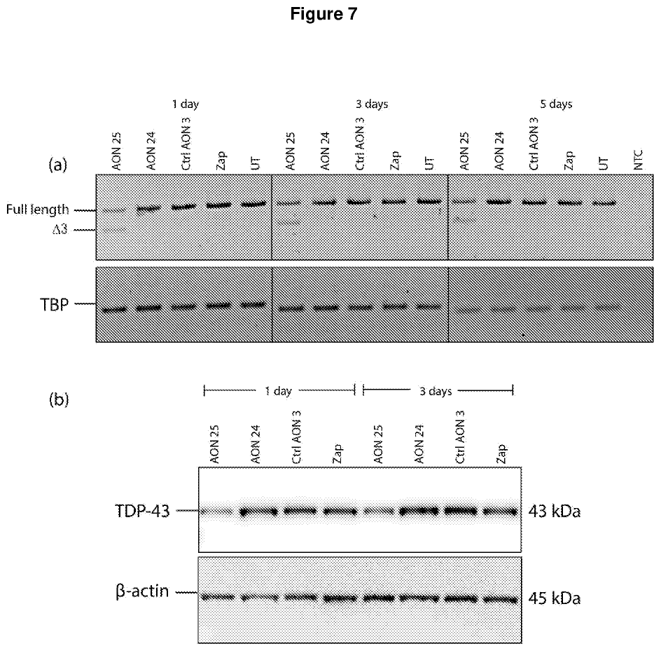

RT-PCR analysis of the TARDBP transcripts is set out in FIG. 7a. PMO AON 24 did not cause knockdown or exon 2 skipping of TARDBP RNA at any timepoint tested. PMO AON 25 did induce skipping of exon 3 and transcript knockdown which was seen at all 3 timepoints and was strongest at 24 hours after transfection. Protein knockdown was also measured by Western Blot as described below.

### Materials and Methods—Western Blotting

Cell lysates were prepared with 125 mM Tris/HCl pH 6.8, 15% SDS, 10% Glycerol, 1.25 μM PMSF (Sigma-Aldrich, NSW, Australia) 1× protease inhibitor cocktail (Sigma-Aldrich) 0.004% bromophenol blue and 2.5 mM dithiothreitol, then sonicated 6 times (1 second pulses). Samples were heated at 94° C. for 5 minutes, cooled on ice and centrifuged at 14,000×g for 2 min before loading onto the gel.

Total protein of approximately 15 μg, measured by a Pierce BCA Protein assay kit (Life Technologies), was loaded per sample on a NuPAGE Novex 4-12% BIS/Tris gel (Life Technologies) alongside a molecular weight marker (Magic Mark) (BioRad) and separated at 200 V for 55 minutes. Proteins were transferred onto a polyvinylidene fluoride (PVDF) membrane at 350 mA for 1 hour. Following blocking for 1 hour at room temperature, the membrane was incubated overnight at 4° C. in 5% skim milk powder in 1× TBST containing TDP43 primary antibody (1:1,000, Proteintech), and Beta-actin primary antibody (1:50,000, Sigma-Aldrich). Immunodetection was performed using an anti-rabbit HRP secondary antibody (1:10,000, Dako) and the Immobilon HRP chemiluminescent substrate (Merck) for TDP43. Immunodetection of Beta-actin was performed using a Western Breeze Chemiluminescent Immunodetection Kit (Life Technologies) with the CDP Star chemiluminescent substrate used for detection. Western blot images were captured on a Vilber Lourmat Fusion FX system using Fusion software and Image J software was used for image analysis.

### Results

The exon 2 targeted PMO (SEQ ID NO: 24) did not cause any TDP43 protein knockdown. Exon 3 targeted PMO (SEQ ID NO: 25) did cause TDP43 protein knockdown at 3 and 5 day timepoints and was greatest at 3 days after transfection with the protein knocked down to 40% of the level seen in control cells when normalised to a housekeeping protein (R-actin) (FIG. 7b).

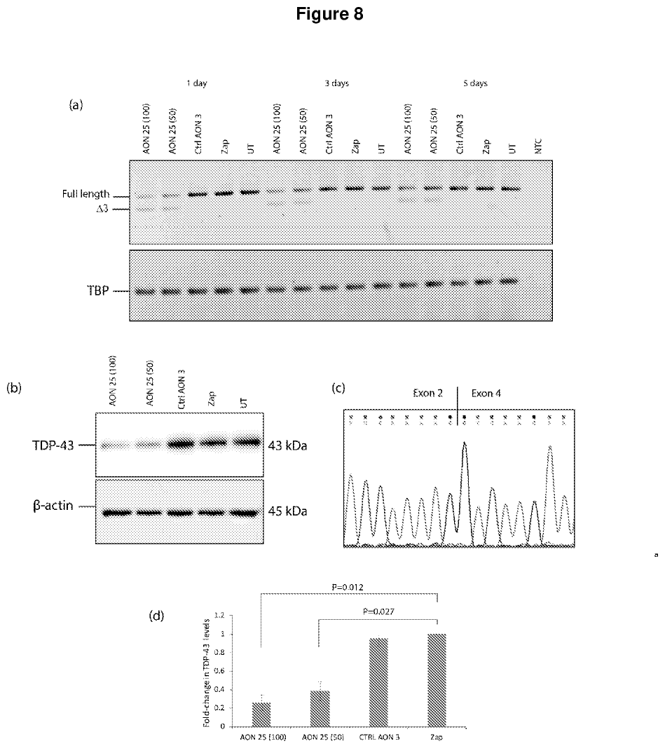

SEQ ID NO: 25 was tested at 100 and 50 μM in 3 more independent experiments using nucleofection as described above with RNA collected at 1, 3 and 5 days and protein collected at 3 days. Exon 3 skipping and knockdown was seen at all 3 timepoints at both concentrations but was highest at 100 μM at 24 hours after transfection (representative gel image FIG. 8a). Exon 3 skipping was confirmed by Sanger sequencing (FIG. 8c). TDP43 levels were knocked down by 74% (p-value 0.012) 3 days after transfection at 100 μM and by 62% (p-value 0.027) at 50 μM compared to control cells that underwent nucleofection with no AON present (Zap) (FIGS. 8b and 8d).

### Example 7—Development of Cell Model to Test STMN2 Upregulation

In order to evaluate the ability of STMN2 cryptic exon targeted AONs to increase stathmin-2 protein levels, a cell model was needed in which stathmin-2 was decreased. In order to mimic the neuronal TDP43 depletion and stathmin-2 downregulation seen in ALS patients, TARDBP exon 3 targeted PMO (SEQ ID NO: 25), which is able to induce TDP43 knockdown via exon 3 skipping was transfected into SH-SY5Y cells using the Neon™ transfection system (Life Technologies).

SH-SY5Y cells were transfected with either 100 or 50 μM of the TARDBP targeted PMO (SEQ ID NO: 25) or 100 μM of a negative control oligo purchased from GeneTools (AON ID 12). All concentrations refer to the concentration in the tip during electroporation. Control cells also underwent electroporation without any AO present (Zap treated cells). Cells were collected for analysis after 1 and 3 days incubation. RNA was extracted from cells and analysed via RT-PCR and agarose gel electrophoresis.

### Transcript Analysis

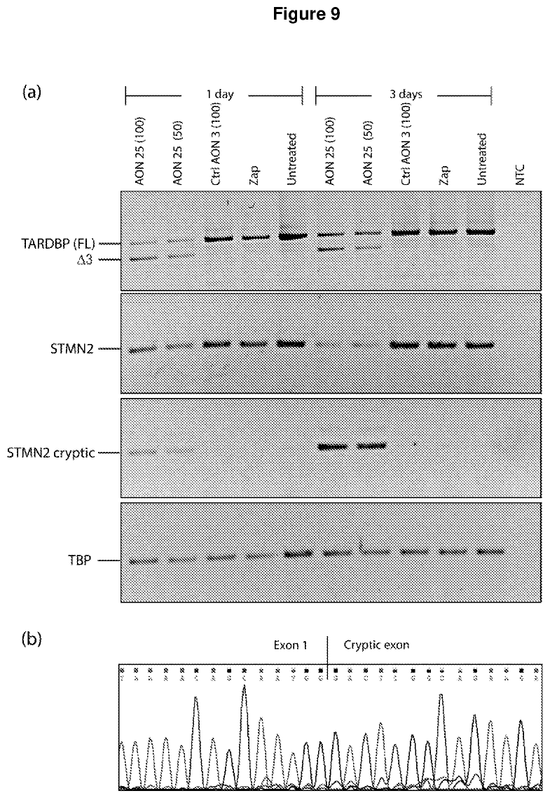

RNA was extracted using the MagMAX-96 Total RNA Isolation Kit, including a DNase treatment (Life Technologies), according to the manufacturer's instructions. RT-PCRs were performed using the One-step Superscript Ill RT-PCR kit with Platinum Taq polymerase (Life Technologies) according to manufacturer's instructions. Products were amplified across TARDBP exons 1 to 6 to measure TARDBP levels/knockdown compared to control and untreated cells (Fwd: CATTTTGTGGGAGCGAAGCG (SEQ ID NO: 67), Rev: ACGCACCAAAGTTCATCCCA (SEQ ID NO: 68)), with the temperature profile, 55° C. for 30 min, 94° C. for 2 min, followed by 24 cycles of 94° C. for 40 sec, 55° C. for 30 sec and 68° C. for 1 min 30 sec. STMN2 levels were measured by amplifying across exons 1 to 3 (Fwd: TGTACTCCAGCACCATTGGC (SEQ ID NO: 71), Rev: AAAGTTCGTGGGGCTTCTGAG (SEQ ID NO: 72)) or 1 to 5 (Fwd: TGTACTCCAGCACCATTGGC (SEQ ID NO: 71) Rev: TGCTTCAGCCAGACAGTTCA (SEQ ID NO: 73)) with the temperature profile, 55° C. for 30 min, 94° C. for 2 min, followed by 28 cycles for 1 to 3 or 26 cycles for 1 to 5 of 94° C. for 30 sec, 60° C. for 20 sec and 68° C. for 1 min 15 sec. As transcripts containing the cryptic exon are polyadenylated at a site within the cryptic exon, STMN2 transcripts containing the cryptic exon were detected by amplifying from exon 1 to a position towards the end of the cryptic exon sequence (Fwd: TGTACTCCAGCACCATTGGC (SEQ ID NO: 71) Rev: GTCAACTGTGCCACAAGCC (SEQ ID NO: 74)) with the temperature profile, profile, 55° C. for 30 min, 94° C. for 2 min, followed by 29 cycles of 94° C. for 30 sec, 60° C. for 20 sec and 68° C. for 1 min. The sequence of the transcript containing the cryptic exon was confirmed via Sanger Sequencing (FIG. 9b). Where applicable, results were normalised to transcript levels of an unrelated housekeeping control gene (TBP) amplified across exons 2 to 3 using the following primers (Fwd: AGCGCAAGGGTTTCTGGTTT (SEQ ID NO: 69), Rev: GGAGTCATGGGGGAGGGATA (SEQ ID NO: 70).

PCR products were fractionated on 2% agarose gels in Tris-Acetate-EDTA buffer and the images captured on gel documentation system (Vilber Lourmat, Eberhardzell, Germany). Densitometric analysis was carried out using Image J. Exon skipping of TARDBP was quantitated by band weight to estimate ratios of full length TARDBP and exon skipped products. Product identity was confirmed by band purification and DNA sequencing as necessary. STMN2 transcript levels were determined by normalisation to a housekeeping gene and comparison to control treated or untreated samples.

TARDBP exon 3 skipping and transcript knockdown was seen at both 1 and 3 timepoints but was strongest 1 day after transfection. Non-cryptic exon containing STMN2 transcripts were knocked down by 80% compared to control treated cells when treated with the TARDBP AON after 3 days incubation. This was concomitant with a large increase in the amount of cryptic exon containing STMN2 transcripts which was strongest at the 3-day timepoint. There was no STMN2 knockdown, nor was the cryptic exon detected in any of the controls. (FIG. 9a).

### Example 8—Design of STMN2 Targeted AONs

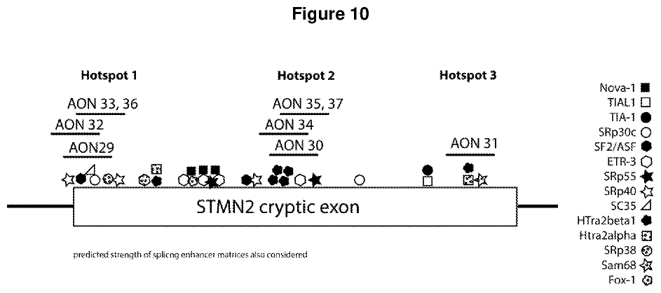

AONs were designed to target sites within the cryptic exon in intron 1 of STMN2. Sites were chosen that would inhibit the binding of splicing enhancers as predicted by online splice prediction tools with AONs targeted to three enhancer site hotspots. The reduced binding of splicing enhancers reduces the recognition of the exon by the spliceosome, leading to the cryptic exon being excluded from the mature mRNA transcript. This would lead to increased levels of stathmin-2 being produced as mature STMN2 transcripts that do not contain the cryptic exon are translated. The AON IDs, sequences, gene co-ordinates, and chemistry can be seen in Table 1. AON binding and enhancer sites can be seen in FIG. 10. AONs with 2′-O-Methyl modifications and a phosphorothioate backbone were ordered from ChemGenes Corporation (Wilmington, MA, USA). AONs with a phosphorodiamidate backbone (PMOs) were ordered from GeneTools LLC (Philomath, OR, USA).

### Example 9—Testing of STMN2 Targeted 2′-O-Methyl AONs

SH-SH5Y cells were transfected with 100 μM of the TARDBP exon 3 skipping PMO (SEQ ID NO: 25) alone or in combination with 5 or 10 μM of one of the STMN2 cryptic exon targeted 2′-O-Methyl-PS AONs (SEQ IDs 29, 30 or 31) or 100 μM of a control oligo (AON ID 28) using the Neon transfection system. All concentrations refer to the concentration in the tip during electroporation. Control cells also underwent electroporation with no AO present (Zap). Cells were collected for RNA extraction and analysis after 1 day and 3 days incubation. RNA was extracted from cells and analysed via RT-PCR and agarose gel electrophoresis with densitometric analysis.

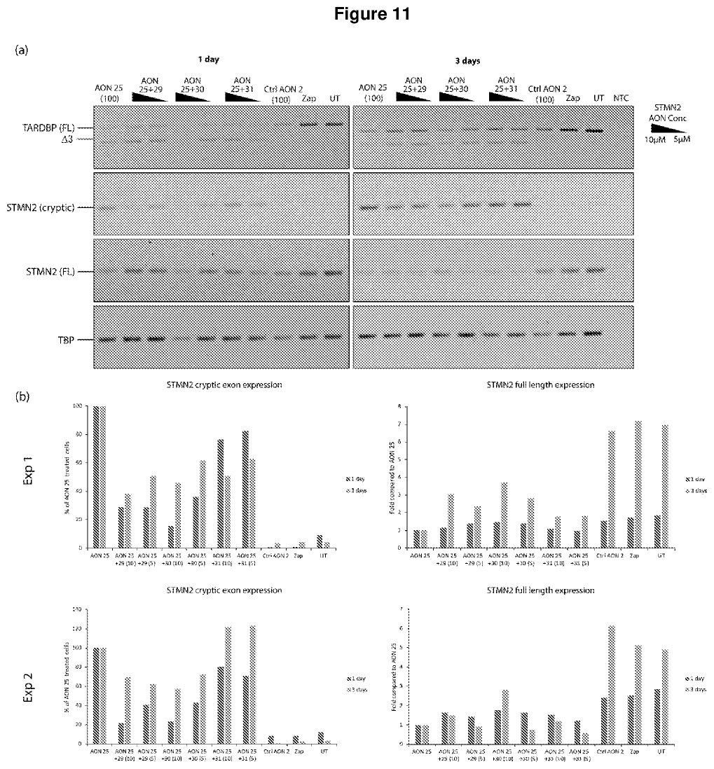

TARDBP was knocked down by approximately 70% after one day in cells treated with the TARDBP exon 3 skipping PMO (SEQ ID NO: 25) compared to controls. STMN2 transcripts containing the cryptic exon were detected in trace levels in control cells but were abundant in cells treated only with SEQ ID NO: 25. This cryptic exon expression was lower in cells that were co-transfected with STMN2 cryptic exon targeted AONs. Compared to cells only treated with SEQ ID NO: 25, levels were reduced to 29% in SEQ ID NO: 29 treated cells, to 15% in SEQ ID NO: 30 treated cells and to 76% in SEQ ID NO: 31 treated cells 1 day after transfection at 10 μM. In cells treated with only SEQ ID NO: 25, levels of the full length STMN2 transcript had reduced to 46% of the levels seen in untreated control cells by 1 day and down to 14% after 3 days. In cells co-transfected with the STMN2 targeted AOs, full length STMN2 levels remained higher with the greatest difference seen at 3 days. In cells transfected with AON 30 (10 μM), full length STMN2 levels were 3.7-fold higher than in cells treated only with SEQ ID NO: 25 after 3 days. Cells treated with SEQ ID NO: 29 (10 μM) had a 3-fold increase and those treated with AON 31 (10 μM), a 1.8-fold increase. A dose response was seen for SEQ ID NO: 29 and SEQ ID NO: 30 with cells treated at 5 μM expressing slightly less of the full length STMN2 transcript. For AON 31, full length STMN2 levels were similar at both concentrations. This experiment was repeated with similar results. Representative gel images can be seen in FIG. 11a and densitometric analysis from both experiments in FIG. 11b.

### Example 10—Optimisation of STMN2 AON Sequences

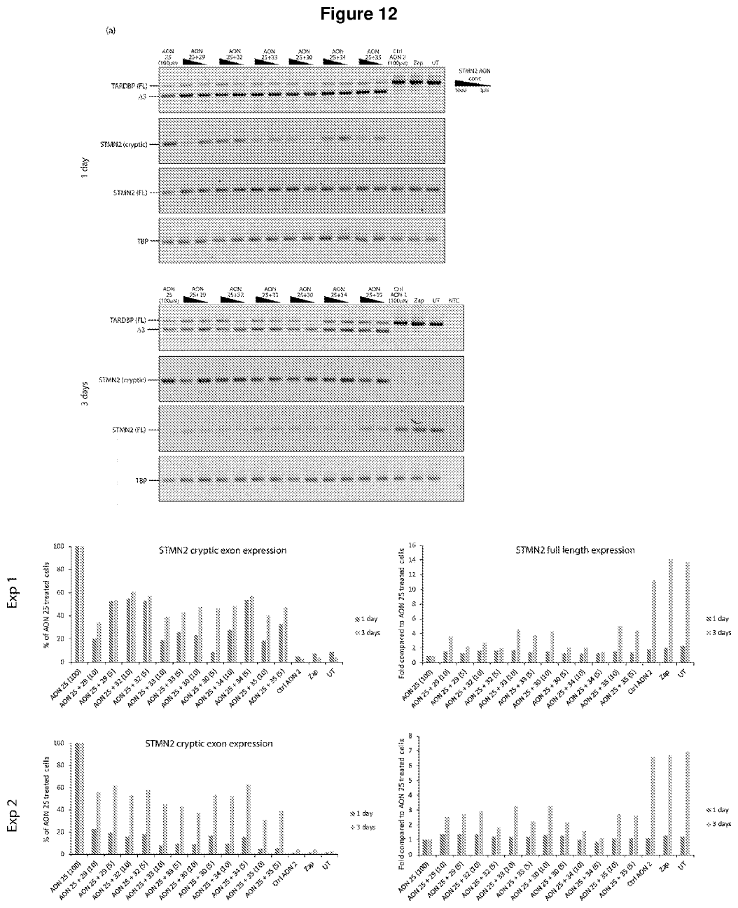

STMN2 AON sequences were optimised by shifting the AON sequence 5 bases upstream and downstream of SEQ ID NO: 29 (SEQ ID NO: 32 and SEQ ID NO: 33) and SEQ ID NO: 30 (SEQ ID NO: 34 and 35). These were tested alongside SEQ ID NO: 29 and SEQ ID NO: 30 in two experiments using the methods previously described. Out of the 3 AONs targeted to the first enhancer site hotspot of the cryptic exon, SEQ ID NO: 33 produced the greatest cryptic exon suppression and the greatest increase in full length STMN2 expression compared to cells transfected with only SEQ ID NO: 25 in two experiments, with STMN2 expression increased up to 4.5-fold. For AONs targeted to the second enhancer site hotspot of the cryptic exon, SEQ ID NO: 30 and SEQ ID NO: 35 produced similar results with full length STMN2 levels at 3 to 5-fold of SEQ ID NO: 25 only treated cells when transfected at 10 μM. At 5 μM SEQ ID NO: 35 co-treated cells had greater full length STMN2 levels than SEQ ID NO: 30 co-treated cells in two experiments. Representative gel images from 1 experiment can be seen in FIG. 12a and densitometric analysis from 2 experiments can be seen in FIG. 12b. SEQ ID NO: 33 and SEQ ID NO: 35 were selected to have synthesised as PMOs (SEQ IDs 36 and 37) for further testing and protein analysis.

### Example 11—Transcript and Protein Analysis Following Co-Transfection with TARDBP and STMN2 Targeted PMOs

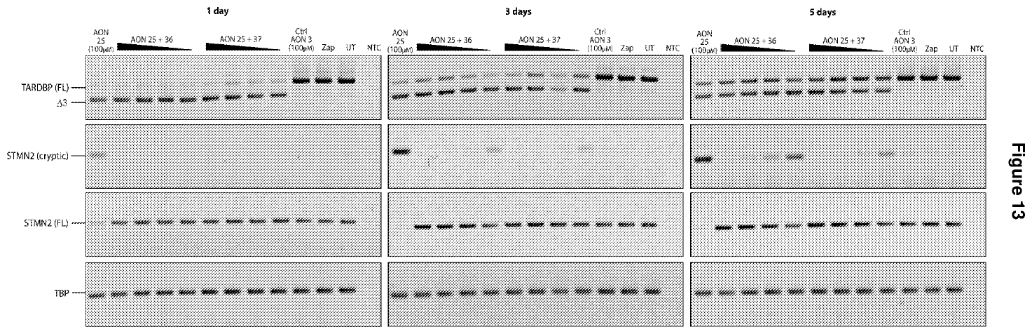

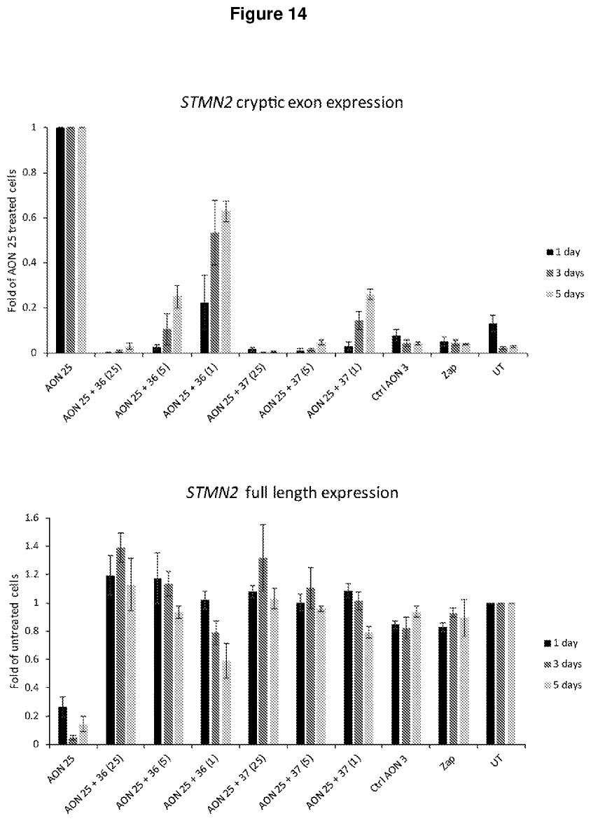

PMO SEQ ID NO: 36 and 37 were tested in SH-SY5Y cells using the same model and methods described above. Cells were transfected with either 100 μM TARDBP exon 3 skipping PMO (SEQ ID NO: 25) or a combination of 100 μM SEQ ID NO: 25 and STMN2 targeted SEQ IDs 36 or 37 at concentrations ranging from 25 μM down to 1 μM (concentration in the neon tip at electroporation) with cells collected for transcript analysis at 1, 3 and 5 days after transfection. The experiment was conducted 3 times. Transcript analysis showed that expression of the cryptic exon, which was greatly upregulated in cells treated with only the TARDBP exon 3 skipping PMO (SEQ ID NO: 25), was supressed by both SEQ IDs 36 and 37 with a clear dose response seen. FIG. 13 shows a representative gel image of RNA transcript analysis. Densitometric analysis from 3 experiments showed that at a transfection concentration of 25 μM both SEQ ID NO: 36 and SEQ ID NO: 37 had supressed expression of the cryptic exon to levels at or below the levels seen in control cells (untreated, Zap and Control AON treated) at all timepoints tested. Five days after transfection at a 5 μM concentration SEQ ID NO: 37 had supressed expression of the cryptic exon to 4.7% and SEQ IDs 36 to 25% of what was seen in TDP43 depleted cells without the addition of an STMN2 targeted AON. Five days after treatment at a 1 μM concentration, SEQ ID NO: 37 co-treated cells expressed 25.9% and SEQ ID NO: 36 co-treated cells 62.9% of the level of cryptic exon containing transcripts as the TARDBP SEQ ID NO: 25 only treated cells (FIG. 14). Densitometric analysis from 3 independent experiments can be seen in FIG. 14a.

In cells treated only with SEQ ID NO: 25, expression of full length STMN2 transcripts was reduced to 14.4% of levels in untreated control cells 5 days after transfection. Expression of full length STMN2 transcripts were maintained at similar levels to control cells when co-transfected with SEQ ID NO: 36 or SEQ ID NO: 37 at 25 μM or 5 μM concentrations. At 1 μM expression of the full length STMN2 transcripts were reduced to 59.2% for SEQ ID NO: 36 and to 79.2% for AON 37 after 5 days (FIG. 14b).

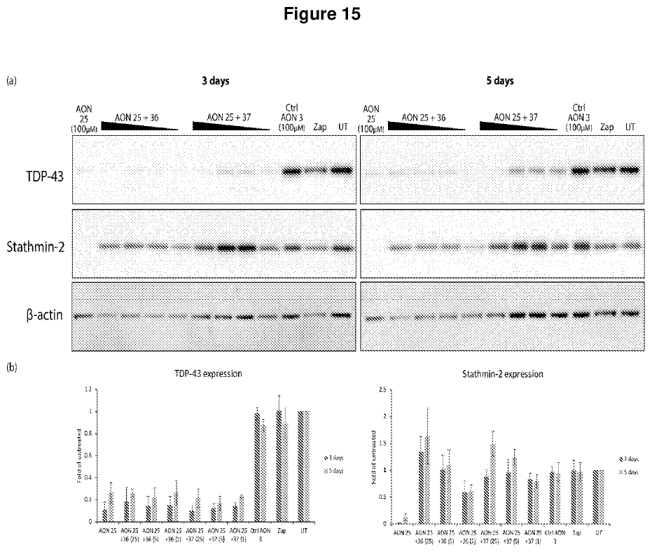

Protein analysis was carried out by Western blot. A representative Western blot image can be seen in FIG. 15a. Densitometric analysis from 3 experiments showed that stathmin-2 expression was completely supressed 3 days after treatment with TARDBP exon 3 skipping PMO (SEQ ID NO: 25) and had risen only slightly by 5 days to 13% of control levels. When cells were co-transfected with the TARDBP targeted SEQ ID NO: 25 and STMN2 targeted SEQ ID NO: 36 or 37 at 25 μM or 5 μM concentrations, stathmin-2 levels were maintained at levels similar to untreated control cells. There was a drop-off in stathmin-2 expression to 60% of control levels at the lowest concentration tested of 1 μM for SEQ ID NO: 25 and SEQ ID NO: 36 co-treated cells 5 days after transfection (FIG. 15b).

### Example 12—Transcript and Protein Analysis Following STMN2 Targeted AON 37 Only

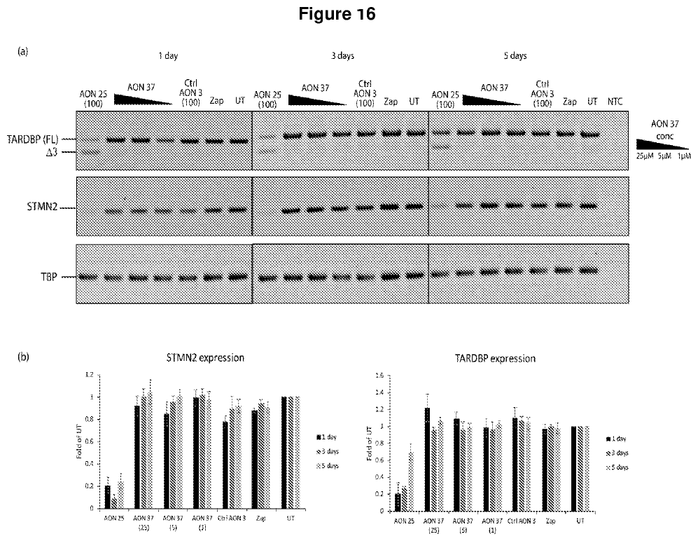

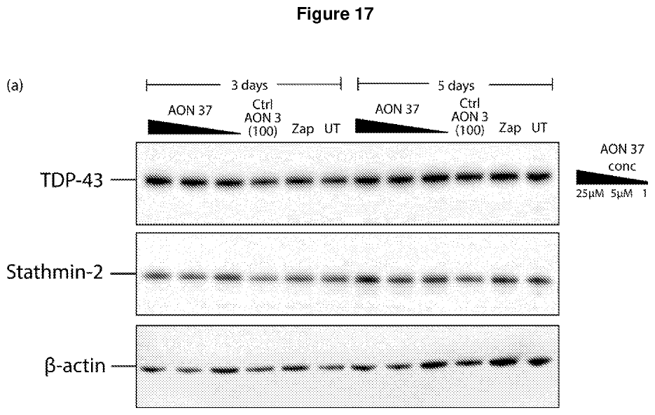

Both under-expression as well as overexpression of stathmin-2 can be deleterious. In order to determine the effect of STMN2 cryptic exon targeted AONs in cells where TDP43 has not been reduced or depleted from the nucleus AON 37 was transfected into SH-SY5Y cells at 3 concentrations in 3 independent experiments. Control cells were transfected with control SEQ ID NO: 3 (SEQ ID NO: 28) or underwent nucleofection with no AON present (Zap). Cells were also transfected with the TARDBP targeted SEQ ID NO: 25 (as a control of transfection efficiency). Transcript analysis via RT-PCR and agarose gel electrophoresis showed that STMN2 transcript levels did not differ significantly from levels seen in control cells after transfection with AON 37 at any time point tested (up to 5 days after transfection). STMN2 levels were reduced to expected levels in SEQ ID NO: 25 treated cells indicating that the transfection efficiency was good. FIG. 16a shows a representative gel image and 16b densitometric analysis from 3 independent experiments. Western blot analysis showed that stathmin-2 protein levels in SEQ ID NO: 37 treated cells remained close to levels seen in control cells after 3 and 5 days (FIG. 17a).

1. An antisense oligonucleotide targeted to a nucleic acid molecule encoding TARDBP pre-mRNA, wherein the antisense oligonucleotide has a nucleobase sequence that is:
a) selected from the list consisting of: SEQ ID NO: 1 to SEQ ID NO: 25, SEQ ID NO: 38 to 58 or a variant thereof; or
b) complementary to at least 1 or more contiguous nucleobases in a target TARDBP pre-mRNA to which SEQ ID NO: 1 to SEQ ID NO: 25, SEQ ID NO: 38 to 58 also binds or a variant thereof,
wherein the antisense oligonucleotide inhibits the expression of the TARDBP gene and
wherein the antisense oligonucleotide is substantially isolated or purified.

2. An antisense oligonucleotide targeted to a nucleic acid molecule encoding STMN2 pre-mRNA, wherein the antisense oligonucleotide has a nucleobase sequence that is:
a) selected from the list consisting of: SEQ ID NO: 29 to SEQ ID NO: 37, SEQ ID NO: 62 to 66 or a variant thereof; or
b) complementary to at least 1 or more contiguous nucleobases in a target STMN2 pre-mRNA to which SEQ ID NO: 29 to SEQ ID NO: 37, SEQ ID NO: 62 to 66 also binds or a variant thereof,
wherein the antisense oligonucleotide prevents the downregulation of and/or increases expression of the STMN2 gene and
wherein the antisense oligonucleotide is substantially isolated or purified.

3. A method of inducing alternative splicing of TARDBP pre-mRNA, the method comprising the steps of:
a) providing one or more of the antisense oligonucleotides according to claim 1; and
b) allowing the oligomer(s) to bind to a target nucleic acid site.

4. A method of inducing alternative splicing of STMN2 pre-mRNA, the method comprising the steps of:
(a) providing one or more of the antisense oligonucleotides according to claim 2; and
(b) allowing the oligomer(s) to bind to a target nucleic acid site.

5. A composition to treat, prevent or ameliorate the effects of a disease associated with TDP43 proteinopathy, the composition comprising:
a) one or more antisense oligonucleotides according to claim 1; and
b) one or more therapeutically acceptable carriers and/or diluents.

6. A pharmaceutical composition to treat, prevent or ameliorate the effects of a disease associated with TDP43 proteinopathy, the composition comprising:
a) one or more antisense oligonucleotides according to claim 1; and
b) one or more pharmaceutically acceptable carriers and/or diluents.

7. A method of treating, preventing or ameliorating the effects of a disease associated with TDP43 proteinopathy, the method comprising the step of administering to the subject an effective amount of the pharmaceutical composition of claim 6.

8. A method for treating, preventing or ameliorating the effects of a disease associated with TDP43 proteinopathy in patients identified by a biomarker, the method comprising the step of:
a) testing a subject for the presence of a biomarker associated with a disease associated with TDP43 proteinopathy patients likely to respond to TDP43 suppression; and
b) if the subject is found to express the biomarker, administering to the subject an effective amount of the pharmaceutical composition of claim 6.

9. A method of reducing the expression of TDP43 in a subject and/or reducing the over expression of TDP43 caused by auto regulation in a subject, the method comprising the step of administering to the subject an effective amount of the pharmaceutical composition of claim 6.

10. A method of preventing the downregulation of the STMN2 gene to maintain normal physiological levels of stathmin-2 and/or increase stathmin-2 expression where it has been reduced in the subject, the method comprising the step of administering to the subject an effective amount of a pharmaceutical composition comprising:
(a) one or more antisense oligonucleotides according to claim 2; and
(b) one or more pharmaceutically acceptable carriers and/or diluents.

11. A method of:
(1) reducing the expression of TDP43 in a subject; and/or
(2) reducing the over expression of TDP43 caused by auto regulation in a subject
and preventing or reducing the downregulation of the STMN2 gene to maintain normal physiological levels or increase expression of stathmin-2 in the subject,
the method comprising the step of administering to the subject an effective amount of a pharmaceutical composition comprising:
(a) one or more antisense oligonucleotides according to claim 1;
(b) one or more antisense oligonucleotides according to claim 2; and
(c) one or more pharmaceutically acceptable carriers and/or diluents.

12. A method of:
(1) reducing the expression of TDP43 in a subject; and/or
(2) reducing the over expression of TDP43 caused by auto regulation in a subject and preventing or reducing the downregulation of the STMN2 gene to maintain normal physiological levels or increase expression of stathmin-2 in the subject, the method comprising the step of administering to the subject an effective amount of:
(a) a pharmaceutical composition comprising one or more antisense oligonucleotides according to claim 1, and one or more pharmaceutically acceptable carriers and/or diluents; and
(b) a second pharmaceutical composition comprising one or more antisense oligonucleotides according to claim 2, and one or more pharmaceutically acceptable carriers and/or diluents,
wherein the two pharmaceutical compositions are administered to the subject concurrently or sequentially.

13. An expression vector comprising one or more antisense oligonucleotides according to claim 1.

14. An expression vector comprising one or more antisense oligonucleotides according to claim 2.

15. A cell comprising the antisense oligonucleotide according to claim 1.

16. A cell comprising the antisense oligonucleotide according to claim 2.

17. The use of antisense oligonucleotides according to claim 1, for the manufacture of a medicament to treat, prevent or ameliorate the effects of a disease associated with TDP43 proteinopathy.

18. The use of antisense oligonucleotides according to claim 1, to treat, prevent or ameliorate the effects of a disease associated with TDP43 proteinopathy.

19. A kit to treat, prevent or ameliorate the effects of a disease associated with TDP43 proteinopathy in a subject, wherein the kit comprises at least an antisense oligonucleotide according to claim 1, packaged in a suitable container, together with instructions for its use.
